(19) United States
(12) Patent Application Publication
(10) Pub. No.: US 2022/0082561 A1
(43) Pub. Date: Mar. 17, 2022

(54) METHODS TO CAPTURE CELLS BASED ON PREFERENTIAL ADHERENCE

(71) Applicant: Worcester Polytechnic Institute, Worcester, MA (US)

(72) Inventors: Seyed Masoud Loeian, Worcester, MA (US); Balaji Panchapakesan, Worcester, MA (US)

(21) Appl. No.: 17/276,329

(22) PCT Filed: Sep. 20, 2019
(86) PCT No.: PCT/US2019/052200

§ 371 Date: Mar. 15, 2021

Related U.S. Application Data
(60) Provisional application No. 62/733,849, filed on Sep. 20, 2018.
(60) Provisional application No. 62/827,577, filed on Apr. 1, 2019.

(51) Int. Cl. G01N 33/551; G01N 1/40; G01N 33/574

(52) U.S. Cl. CPC G01N 33/551; G01N 2015/1006; G01N 33/574; G01N 1/4077

(57) ABSTRACT

Methods and devices are provided for the detection and characterization of circulating cells in a blood sample. Such method can include depositing a sample of a bodily fluid on a device comprising carbon nanotubes, wherein the surfaces of the carbon nanotubes are not functionalized; and detecting target cells adhered to the carbon nanotubes.

### CROSS-REFERENCE TO RELATED APPLICATION

This application claims the benefit of the following U.S. Provisional Application Nos.: 62/733,849, filed Sep. 20, 2018, and 62/827,577, filed on Apr. 1, 2019, their entire contents of which are incorporated herein by reference.

### TECHNICAL FIELD

The disclosure relates generally to droplet biopsy chips for adherence-based capture of circulating cells.

### BACKGROUND

The classic hallmarks of a tumor with metastatic potential include mobility and invasiveness. Metastasis occurs when tumor cells from a primary organ are shed into the vasculature/lymphatics and carried to a distant site, where conditions are conducive for their proliferation. During this process, the circulating tumor cells (CTCs) change morphology, chemical composition, acquire the ability to overcome the defenses of the immune system, the shear stress present in the circulatory system, and programmed cell death due to the lack of extracellular interactions in circulation. CTCs are rare, comprising as few as 1-10 cells per 10⁹ hematological cells, and CTC shedding from a solid tumor into the bloodstream is a highly discontinuous process. Thus, the isolation of CTCs with high purity is still a very significant challenge. In addition to single CTCs in circulation, CTCs have also been observed in clusters, with micro-tentacles, and having multiple phenotypes, and these less understood CTCs are also believed to be metastatic initiators. Thus, capturing and studying CTCs with biomarker heterogeneity at the single cell level could shed light into the complex biological processes at work and enable dynamic views of cancer metastasis. It could also potentially save lives as identification of subset of CTCs with metastatic phenotypes among primary tumor cells in early stage cancer can result in customized therapeutic intervention that could result in better outcome (ex: small number of EGFR+ CTCs among group of CK+ CTCs).

Technologies for CTC capture and enumeration can be broadly classified into immunoaffinity (antigen-dependent) based capture and capture based on cellular physical properties (antigen-independent; ex: size, deformability, cell surface charge, and density). However, all of these techniques have many shortcomings and challenges.

Microfluidics has emerged an active field of research for isolation of CTCs. Microfluidic technologies such as polymer fluidics, CTC-Chip, Herringbone chip, CTC-iChip, Vortex, Accucyte, Fluxion, NanoVelcro, DEP-Array, Parsotrix and JETTA are fluidic devices that has been demonstrated to capture CTCs. Howver, these methods are thighly time-consuming, labor intensive, serial production processes and can enable false positive or negative results thereby severely restricting their applicability to routine clinical practice.

Accordingly, there is a need for methods and systems for capture and isolation of CTCs from bodily fluid samples.

### SUMMARY OF THE INVENTION

As described below, the present disclosure features methods and compositions directed to detecting cells in a sample.

In one aspect a method for capturing target cells in a blood sample is provided that involves removing red blood cells from the sample, depositing the sample on a device comprising one or more carbon nanotubes arrays, wherein the surfaces of the carbon nanotubes are not functionalized; and detecting target cells adhered to the carbon nanotubes.

Another aspect provides a method of detecting circulating tumor cells in a subject suspected of having cancer or previously diagnosed with cancer involving removing red blood cells from a sample derived from the subject, depositing the sample derived from the subject on a device comprising one or more carbon nanotubes arrays, wherein the surfaces of the carbon nanotubes are not functionalized, and detecting the presence or absence of circulating tumor cells adhered to the carbon nanotubes, wherein the presence of circulating tumor cells is indicative of cancer.

Some embodiments of these methods also involve removing serum from the sample prior to depositing the sample on the device comprising carbon nanotubes. In some embodiments, removing red blood cells involves lysing the red blood cells. In some embodiments, removing the red blood cells comprises centrifuging the sample. In some embodiments, the methods also involve removing non-adhered cells or material from the device comprising carbon nanotubes. In some embodiments, the non-adhered cells include white blood cells.

In another aspect, a method is provided for capturing target cells in a sample of a bodily fluid comprises removing from a sample of a bodily fluid cells having a settling rate higher than a settling rate of target cells; depositing the sample on a device comprising one or more carbon nanotubes arrays, wherein the surfaces of the carbon nanotubes are not functionalized; and detecting target cells adhered to the carbon nanotubes.

In some embodiments of any of the above methods, the methods also involve counting the cells adhered to the carbon nanotubes. In some embodiments, the methods also involve adding culture medium to the sample, wherein the culture medium is selected based on the target cell or circulating tumor cell. In some embodiments, the methods also involve removing non-adhered cells or material from the device comprising carbon nanotubes. In some embodiments, the methods also involve washing the device to remove any non-adhered cells. In some embodiments, the non-functionalized carbon nanotubes are coated in collagen. In some embodiments, a surface of the device has an SU8 layer. In some embodiments, the device is disposed on the surface of an array. In some embodiments, the methods also involve collecting the target cells adhered to the carbon nanotubes. In some embodiments, the methods also involve characterizing the collected target cells. In some embodiments, characterizing comprises genotyping the collected target cells, phenotyping the collected target cells, or both. In some embodiments, phenotyping comprises determining the epithelial, mesenchymal, and epithelial to mesenchymal transition states of the collected target cells. In some embodiments, the amount of time elapsed between depositing the sample on the device improves the capture rate of target cells. In some embodiments, between 1 and 1500 target cells or circulating tumor cells are captured per milliliter of blood. In some embodiments, the density of the carbon nanotubes is between 1 and 5 nanotubes per micrometer.

### BRIEF DESCRIPTION OF THE DRAWINGS

FIGS. 1A-1H illustrate carbon nanotube (CNT) devices and arrays of the present disclosure. FIG. 1A is a schematic of multiple (CNT) devices. FIG. 1B is an image of a droplet biopsy microarray comprising multiple CNT devices. FIG. 1C is a diagram depicting the steps in isolation and enumeration of CTCs using Nanotube—Chip. FIG. 1D is a scanning electron micrograph (SEM) image of nanotubes. FIG. 1E is an atomic force microscopy (AFM) image of nanotube film. FIG. IF is an optical image of a wafer comprising a 76-element array; the individual devices (blank and blood adsorbed) are shown in the inset. FIG. 1G is a top view image of a carbon nanotube device according to some embodiments of the present disclosure. FIG. 1H depicts the Raman spectrum of carbon nanotubes, which is characterized by a small D band, large G band, and pronounced 2D band.

FIG. 2A is a diagram of the steps in isolation and enumeration of CTCs using a CNT chip.

FIG. 2B is a flow chart depicting the steps of an exemplary method of the present disclosure.

FIG. 3A is a graph depicting the capture efficiency of adhered versus non-adhered spiked cells in blood.

FIG. 3B comprises graphs depicting the tracking number of adhered cells in each individual device.

FIG. 3C is a fluorescent image of adhered MDA-MB-231 cells versus WBCs (DAPI only).

FIG. 4A is an SEM image of a single SKBR3 breast cancer cell attached on the nanotube surface; inset: high magnification showing 150-200 nm filaments from the cell body attaching to the nanotube surface.

FIG. 4B is a graph depicting the optimization of adherence suggesting 48 hours is the optimized time for cell attachment.

FIG. 5A comprises a graph depicting the capture efficiency of spiked cells using collagen adhesion matrix scaffolding.

FIG. 5B comprises graphs depicting the number of cells counted in each droplet for each spiking experiment.

FIG. 6 comprises optical, DAPI, CK8/18, CD45, and merge images of cells from breast cancer patient and healthy control. CTCs are often no larger than WBCs and the image illustrate this. All scale bars are the same.

FIG. 7 is a set of images of heterogeneous CTCs isolated from breast cancer patients based on CK, Her2, and EGFR. No CTCs were found in healthy controls. The volume of blood: 4 ml and 8.5 ml blood. CK+, CD45− and DAPI+ was identified as CTCs, while CD45+ and DAPI+ identified as WBC.

FIG. 8A is a merge image of CK+, EGFR+, and DAPI+ cells on the same chip; the cell at the bottom is a single cell expressing both CK and EGFR suggesting heterogeneous CTC phenotypes exists.

FIG. 8B is an image of spindle-shaped partial epithelial and partial mesenchymal cell expressing both CK and EGFR.

FIG. 8C is an image of fully epithelial CTC, WBC and mesenchymal CTCs (expressing only EGFR and not CK8/18).

FIG. 8D is an image of epithelial CTC expressing no EGFR and only CK8/18.

FIG. 9A is a fluorescence microscopy image of blood droplet with MDA-MB-231-GFP cells at different depth of focus.

FIG. 9B is a graph showing the number of GFP observations versus number of cells spiked.

FIG. 9C is a graph showing the number of observed cells in each droplet. The observations indicated anywhere from 87-100% capture is possible. Slight errors in cell count at lower concentration is a result of spiking using a hemocytometer.

FIG. 10 is a fluorescence microscopy image of the entire droplet with MDA-MB-231-GFP cells.

FIG. 11A is a diagram showing an RBC lysis protocol.

FIG. 11B comprises images of a blood smear before and after lysis.

FIG. 11C is a fluorescent microscopy image of adhered versus non-adhered cells.

FIG. 12A is an image of U-251, U-343, and LN-229 cells adhered to the nanotube surface.

FIG. 12B is an image of HeLa cells attached to the nanotube surface stained for CD59.

FIG. 13 comprises optical images and merge images of patient samples.

FIG. 14 comprises images depicting heterogeneous CTCs and WBCs on the same chip; optical, DAPI, EGFR, CK and merge images. A single CTC is seen at the bottom of each image suggesting this CTC was positive for DAPI, CK, EGFR suggesting multiple phenotypes on the same cell.

FIG. 15 comprises images depicting epithelial, mesenchymal and EMT related CTCs along with WBCs (DAPI only).

### DETAILED DESCRIPTION

The present disclosure provides methods and devices for producing and using a droplet biopsy chip to capture target cells (e.g., circulating tumor cells or CTCs). These methods and devices provide preferential adherence of CTCs, antigen and size independent capture; 5-6 log depletion of WBCs; with CTCs of multiple phenotypes. Capturing target cells can inform diagnosis and prognosis of a patient, and the number, topography, and genetic information of captured cells can be used to optimize patient treatment. The methods and devices of the present disclosure merely require a sample of a bodily fluid (e.g., blood, lymph, urine, saliva, and the like) from a patient. In some embodiments, using the devices and methods discussed herein, circulating cells adhere to a nanotube surface. For example, target cells in a blood sample will adhere to a nanotube surface after removal of the red blood cells (RBCs) and serum from the sample. In some embodiments, the removed RBCs and serum are replaced with appropriate culture medium volume. In some embodiments, the samples are incubated in a controlled environment. Targeted cells (i.e., CTCs) adhere to a nanotube surface while other elements (e.g., leukocytes) in blood do not. Gently washing the nanotube surface removes non-adhered cells and the captured target cells can be enumerated.

The present disclosure provides a new method of CTC capture based on microarrays of carbon nanotube (CNT) surfaces. This technique is a new type of antigen-independent capture, where the preferential adherence of CTCs to a CNT surface is exploited. The present method can have one or more of many advantages, such as, 1) microarray format enabling a large volume of blood to be RBC lysed/fractionated into smaller portion that may enable better capture sensitivity from droplets; 2) antigen-independent capture of CTCs enables isolation of CTCs of variable phenotypes; 3) size-independent capture of CTCs; 4) the preferential adherence of CTC to nanotube surface enables 5-log depletion of white blood cells (WBCs); 5) no transfer of CTCs is necessary for subsequent microscopy, eliminating cellular loss; 6) planar surface architecture eliminates imaging problems and large image files associated with imaging CTCs inside a fluidic chamber; 7) surface architecture lends itself to easier CTC downstream analysis, unlike microfluidics, where CTCs may be recovered from sealed chambers; and 8) planar batch manufacturing process resulting in >99% yield of individual devices both in silicon-based and glass based wafers.

In some embodiments, the present methods result in a preferential attachment of cancer cells at 89-100% capture rate, isolation of CTCs with high purity 0 and 100% sensitivity (n=7/7) in breast cancer patients (4 ml and 8.5 ml blood), and capture of single CTCs of multiple phenotypes from the same patient. In some embodiments, between 1 and 1500 CTCs are captured per milliliter of blood. In some embodiments, between 500 and 1500 CTCs are captured per milliliter of blood. In some embodiments, between 1000 and 1500 CTCs are captured per milliliter of blood. The microarray format, use of carbon nanotubes for capture based on adherence, and the successful isolation of CTCs of different phenotypes demonstrate that the nanotube-CTC-chip is a versatile platform to capture CTCs in patients.

### Carbon Nanotube Devices

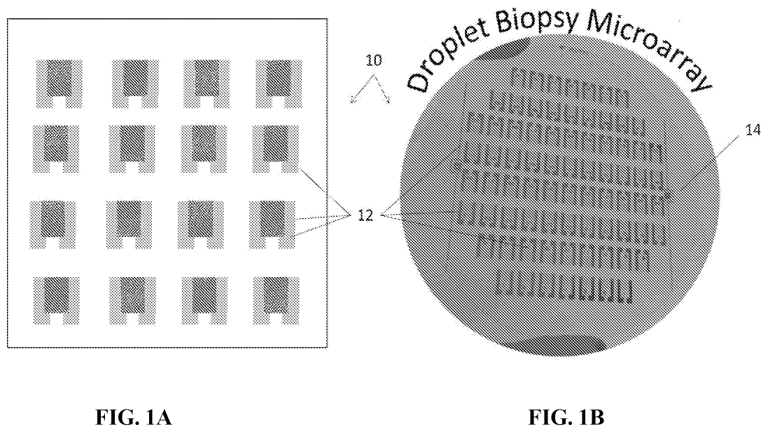

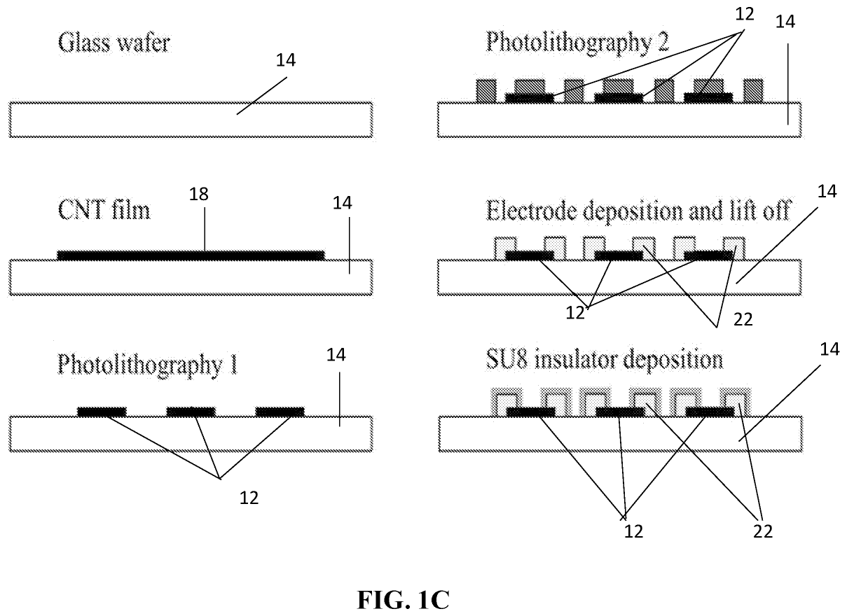

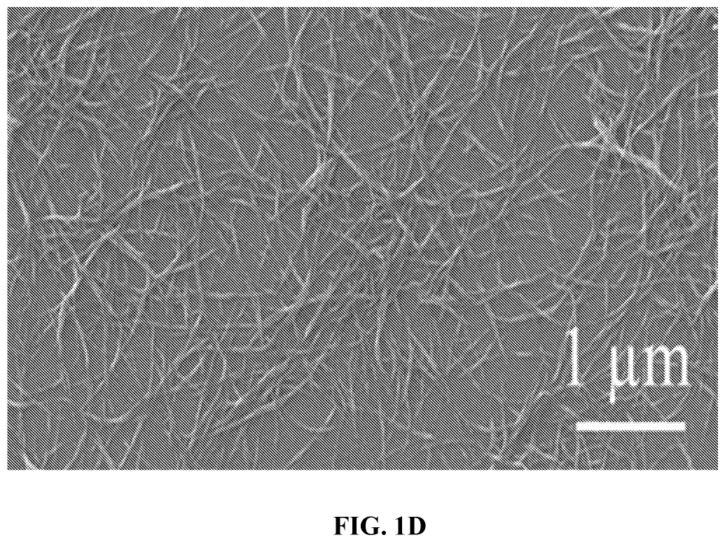

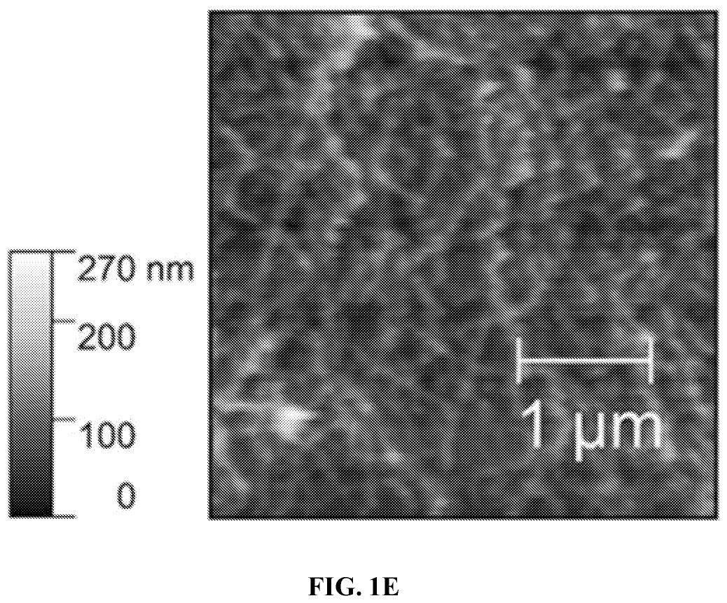

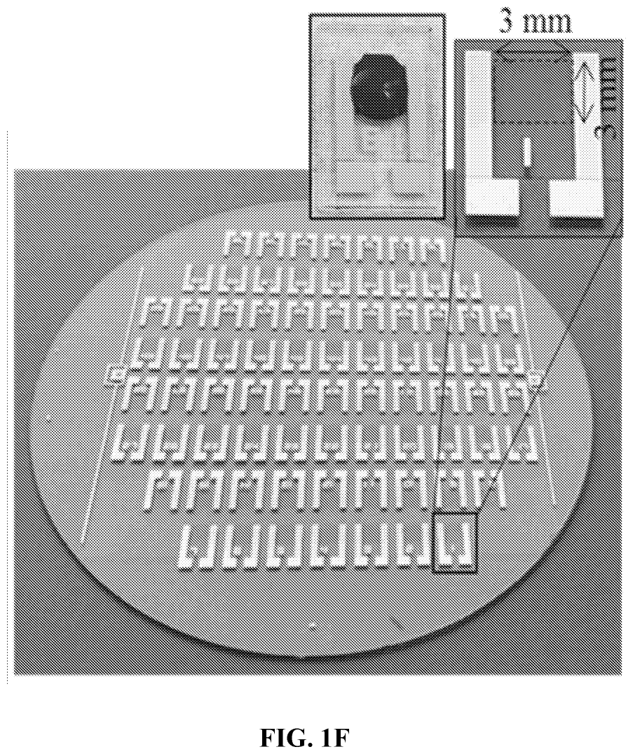

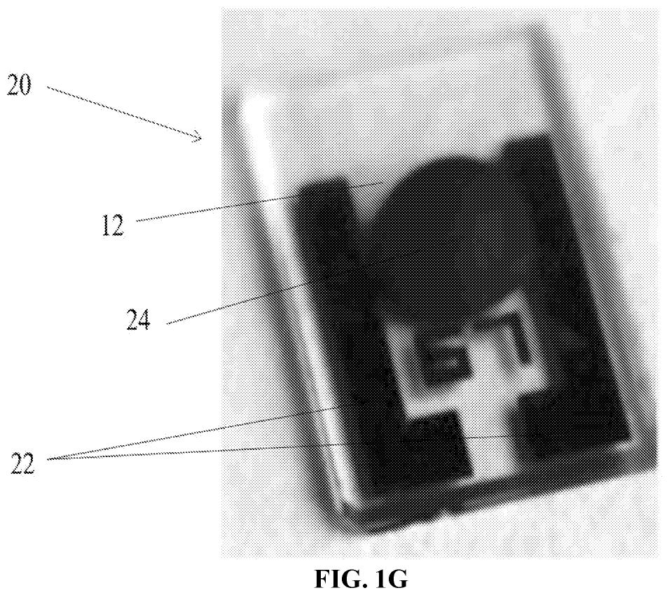

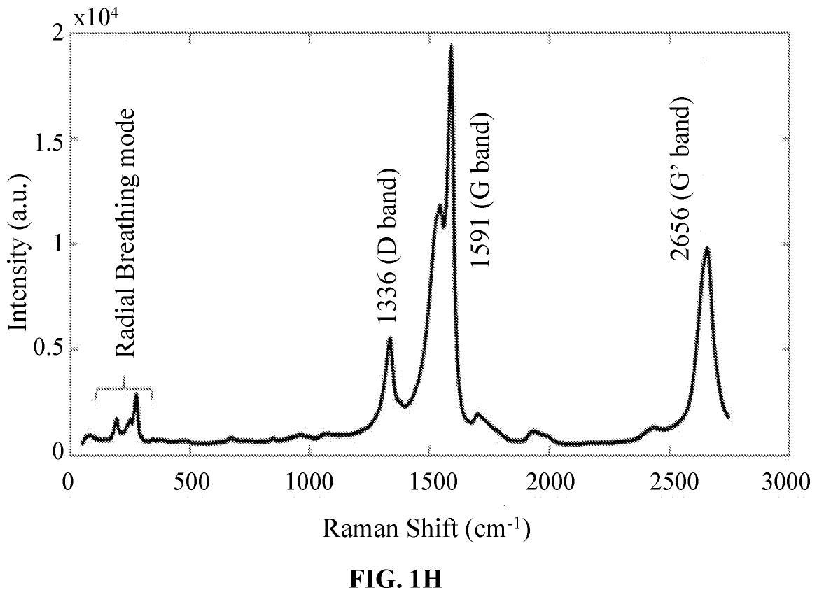

The present disclosure describes capturing cells in a sample using carbon nanotube micro-arrays. In some embodiments, the carbon nanotube devices do not comprise antibodies for capturing targeted molecules or cells. In some embodiments, the carbon nanotube device does not comprise electrodes. FIGS. 1A and 1B depict carbon nanotube (CNT) arrays 10 (also referred to as CNT chips and droplet biopsy microarrays) comprising multiple CNT devices 12 for non-evasive detection and capturing cells (e.g., for diagnosis). In some embodiments, the chip or array comprises, 2, 3, 4, 5, 6, 7, 8, 9, 10, 20, 30, 40, 50, 60, 70, 80, 90, 100, or even more individual CNT devices. An array comprising 10 CNT devices is referred to as a 10-element array. In some embodiments, the chip includes a 76-element array of carbon nanotube devices, as depicted in FIG. 1B. The size of the array can vary based on the size of the wafer 14. For example, a four-inch wafer can have 76 CNT devices, a six-inch wafer can have 100 CNT devices, a twelve-inch wafer can have 130 CNT devices. Different number of arrays and devices of different size can be provided. In this manner, each device in an array is able to capture a large number of cells. In some embodiments, the number of devices is increased or decreased depending on the volume of blood to be processed by the chip. The blood can be applied using a pipette. In some embodiments, a saline solution is applied to a device and then a sample (e.g., blood) is applied to the saline solution.

In some embodiments, electrodes are utilized on the CNT devices. In some embodiments, the electrodes are employed to deliver an electrical stimulation to the cells. In some embodiments, the electrodes are used to measure a change in electrical conductivity to detect the capture of target cells by the carbon nanotubes of the CNT devices.

The CNT devices may be formed using a variety of techniques. In some embodiments, the array of CNT devices 12 can be fabricated on a glass wafer 14, as depicted in FIG. 1C. In some embodiments, vacuum filtration is used for creating a CNT film 18 and transferring the CNT film 18 onto glass wafer 14. This is an effective way to fabricate macroscopic CNT thin films 18 with random orientation and filtration process self-regulate the distribution of the CNT devices 12 on the wafer 14. By controlling the concentration of stock solution, it is possible to control the density of the CNT film and thus CNT devices 12. In some embodiments, the weight of CNT devices 12 on each wafer 14, before the fabrication process, can be about 100 μg. The weight can range from about 1 to about 1000 μg based on the design of the CNT devices 12 and the number of devices in the array. In some embodiments, the height of CNT device 12 bundles ranges between about 100-200 nm.

Referring again to FIG. 1C, which shows an exemplary microfabrication process for making the CNT devices and CNT thin films discussed herein, a wafer (e.g., glass or silicon) is provided as a substrate for CNT devices (I). A CNT film is then deposited on the wafer (II). A first photolithography step followed by masked patterning of the CNT film and reactive-ion etching (RIE) creates the device pattern in shown in (III).

In the embodiments with electrodes, after removing the first mask (e.g., with acetone), a second mask can be patterned using a second photolithography step on the devices for the fabrication of electrodes (IV). In particular, desired areas of the CNT film can be covered with a photoresist using photolithography in a clean room. Thereafter, the rest of the CNT film can be etched away using oxygen plasma in a reactive ion etcher for 120 s at 200 W and 200 mTorr (V). The duration and pressure of the etching process can be chosen based on the weight of the CNT devices. In some embodiments, an about 15 nm thick nickel layer and an about 90 nm thick gold layer is deposited on CNT areas by sputtering. The nickel layer provides an adhesion layer between the gold layer and the CNT devices. The gold layer should be sufficiently dimensions to make sure it will not wear off and lose its performance as an electrode. Alternative materials can be utilized in place of nickel and gold without departing from the scope of the present disclosure. For example, chromium can be used in place of nickel, or a chromium nickel alloy can be used as an adhesive layer for the gold layer (or other conductive material). In some embodiments, sputtering Ni/Au and mask removal (e.g., with acetone) creates the electrodes (V). Removing the second mask is the last fabrication step, and the devices are diced using a wafer dicing saw for the application.

In some embodiments, a thick SU8 (an epoxy-based negative photoresist) layer not only isolates the electrodes but also contains the droplet only on the CNT film area in each chip (VI). The SU8 layer is used for passivation, and the liquid only is exposed to the carbon nanotube area and not the electrode area. FIG. 1C presents a non-limiting example of one fabrication process. Other processes known in the art can also be used.

In some embodiments, the CNT thin films are formed from a single layer of semiconducting nanotubes. In some embodiments, single wall carbon nanotubes may be employed. In some embodiments, the nanotubes have a purity higher than 90%, or, in some embodiments, higher than 95% or higher than 99.5%. In some embodiments, the density of the nanotubes is between 1 and 5 nanotubes per micrometer. In some embodiments, the density is between 3 and 5 nanotubes per micrometer. In some embodiments, the density is 5 nanotubes per micrometer. In some embodiments, the density is controlled through a filtration process using a known concentration of nanotubes in the starting material. In some embodiments, the nanotubes are deposited in a single layer.

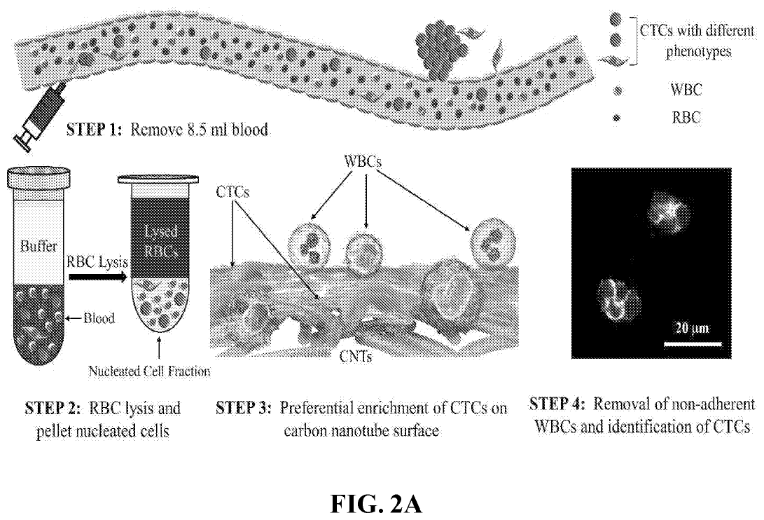

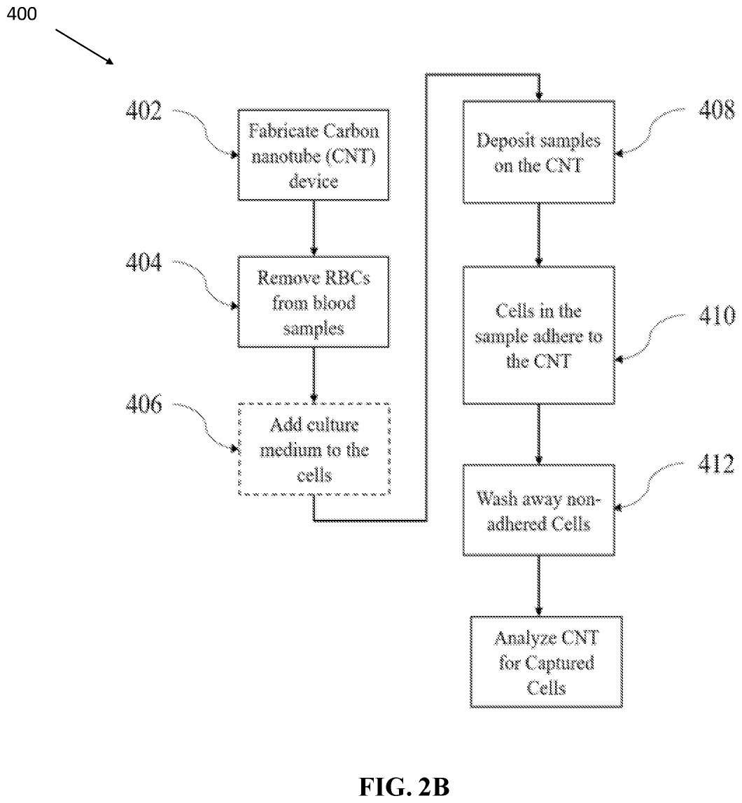

FIGS. 1D and 1E show a scanning electron microscopy (SEM) and atomic force microscopy images, respectively, of a random distribution of CNT devices distributed on a glass wafer. FIG. 1F is a photograph image of an array comprising multiple CNT devices, with insets showing the dimensions of each device on the array (3×3 mm) and a device loaded with a blood sample. FIG. 1G is a close-up of a device chip 20 comprising a CNT device 10 that is loaded with a blood sample 24 between two electrodes 22. FIG. 1H is the Raman spectra of an exemplary CNT film on a glass wafer. The first peak (i.e., the Radial Breathing Mode (RBM)) is observed around 250 cm⁻¹. RBM is due to radial expansion-contraction of the nanotube. Therefore, its frequency, V_{RBM} (CM⁻¹), is a function of nanotube diameter (d) (V_{RBM}=A/d+B, where A and B are constants for single wall CNT, B is estimated as 0 and A is estimated as 248 cm¹. In FIG. 2D, the frequency of RBM is 275.8 cm¹, and therefore, the diameter of single wall CNTs are roughly estimated as 0.9 nm. As a result of a defective activated band in sp2 hybridized carbon materials, D band is observed and its frequency is 1338 cm⁻¹. The first-order Raman band of all sp2 hybridized carbon materials (the “G band”) is 1590 cm⁻¹. The ratio of D and G bands provides information about the defects in the CNT devices.

### Preferential Adherence-Based Cell Capture

Preferential adherence refers to the concept that CTCs preferentially attach to nanotube surfaces, but other blood components, including white blood cells (WBCs), do not. The present method includes using a bare or non-functionalized carbon nanotube surface. As used herein, “non-functionalized,” refers a carbon nanotube surface that does not comprise any compound, ligand or protein or fragment thereof that comprises a binding domain or moiety having an affinity for a specific cellular antigen. For example, a carbon nanotube surface having one or more antibodies conjugated thereto would be a functionalized CNT surface capable of binding only those cells that express a specific antigen recognized by the antibodies. In contrast, the devices described herein utilize bare or non-functionalized carbon nanotube surfaces to capture adherent cells in a sample. For example, in some embodiments of the present disclosure, a device comprising bare or non-functionalized carbon nanotube surfaces is used to capture epithelial breast tumor cells in a blood sample that adhere to the carbon nanotube surfaces.

In some embodiments, a carbon nanotube surface is coated with collagen. Such a surface is considered to be a non-functionalized surface because collagen does not specifically bind to any particular cell or antigen expressed on a cell.

In some embodiments, these devices are made on glass wafers. In some embodiments, the devices are made on a silicon wafer. The method is applicable to all major epithelial cancers namely breast, prostate, lung, and colon cancer.

The isolation, capture, and enumeration of CTCs of different phenotypes using the methods described herein can be broadly characterized as having 4 steps (FIG. 2A). In step 1, blood (for example, consisting of approximately 40 billion erythrocytes, 64 million leukocytes and 1-10 CTCs of different phenotypes) is drawn from a patient. In step 2, the red blood cells (RBCs) are depleted through an RBC lysis protocol. The sample is can be centrifuged, and nucleated cell fractions consisting of CTCs and WBCs pelleted. In step 3, the nucleated cells consisting of CTCs and WBCs are added to the devices. For example, CTCs and WBCs can be added as standard 10-20 μl droplets on 6-12 individual nanotube devices (total 60-120 μl) In some embodiments, the dimensions of each device range from 3 mm×3 mm to 8 mm×8 mm. In some embodiments, CTCs, but not other nucleated cells including WBCs attach to the nanotube substrate. Also called antigen-independent CTC capture, this strategy enriches of CTCs with high purity that is not biased by the selection of potentially variably expressed markers on tumor cells. In step 4, the attached CTCs on the nanotube surface can be immune-stained on-chip using antibodies to identify and enumerate CTCs of different phenotypes. DAPI (4′,6-diamidino-2-phenylindole) is used as the nuclear stain and cytokeratin (CK8/18) and other antibodies (ex: Her2, EGFR) identify CTCs.

FIG. 2B shows an exemplary method 400 of the present disclosure. At step 402, a carbon nanotube film is fabricated into one or more CNT devices, as discussed with respect to FIGS. 2A-2E. At step 404, red blood cells (RBCs) and serum are removed from a blood sample by a lysis and centrifugation process. RBCs have a propensity to settle more quickly than other cell types due to their iron content, which leads to its greater density compared to other cell types. By settling, the RBC, being more numerous than CTCs or other target cells, are more likely to interfere or compete with target cell binding to the nanotube surfaces. During step 406, cells are resuspended in a small volume of culture medium. The culture medium can be selected based on the type of cells being targeted for capture. In some embodiments, steps 404 and 406 are implemented with a fresh blood sample or within 4 to 8 hours of sample collection. Alternatively, the blood sample can be stored (e.g., at 4 degrees Celsius).

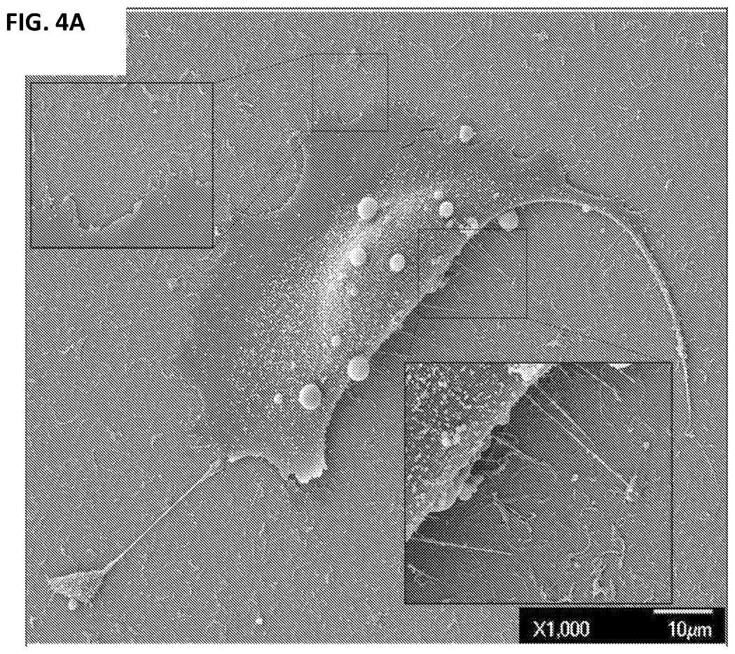

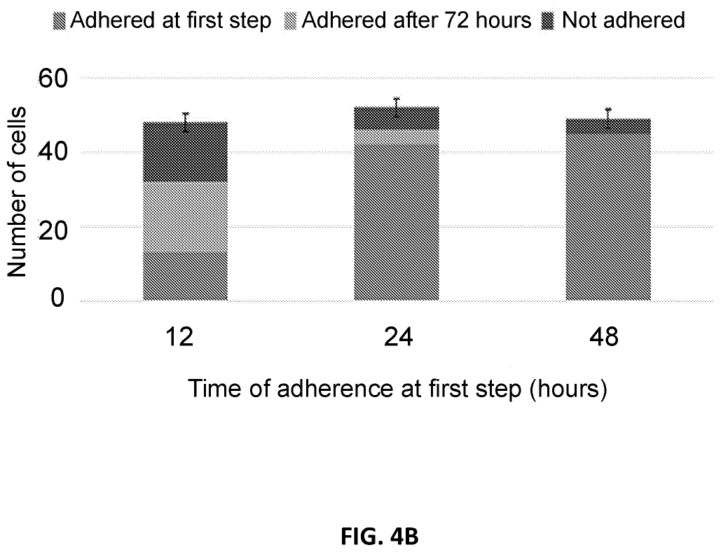

At step 408, the sample having been processed to remove RBCs, is deposited onto the CNT device. During step 408, the sample on the device is incubated, which allows target cells in the sample to adhere to the surface of the CNT films of the CNT device. In some embodiments, incubation is 24 to 36 hours. At step 412, the surface of the device is washed to remove non-targeted elements, leaving viable target cells attached to target surface for further processing. In some embodiments, the washing can include gently aspirating the droplet from the surface of the device and adding a 10 μl droplet of PBS to wash the surface. Following the process 400 diagrammed in FIG. 4, target cells can be captured by the CNT film without pre-labelling, pre-fixation, or any other processing steps. In some embodiments, before spiking, it is possible to count the cells using hemocytometer and an optical microscope. After completing the process of capturing cells and washing the device, the cells should be stained with proper biomarkers in order to count them and confirm their viability under fluorescent microscope. In some embodiments, DAPI is used to stain nuclei. In some embodiments, fluorescently labelled antibodies that bind cytokeratin are used to count cancer cells.

In operation, a blood sample comprising target cells is deposited onto a diagnostic device of the present disclosure, as depicted in FIG. 1G. In some embodiments, red blood cells (RBCs) and serum are removed from a blood sample by a lysis and centrifugation process and resuspended in a small volume of culture medium. The culture medium can be selected based on the type of cells being targeted for capture. In some embodiments, the blood sample is a freshly drawn sample or within 4 to 8 hours of sample collection. Alternatively, the blood sample can be stored (e.g., at 4 degrees Celsius).

In some embodiments, a blood sample processed to remove RBCs is deposited onto a CNT device and incubated, which allows target cells in the sample to adhere to the surface of the nanotubes of the CNT device. In some embodiments, incubation is 24 to 36 hours. In some embodiments, the surface of the device is washed to remove non-targeted elements, leaving viable target cells attached to target surface for further processing. In some embodiments, the washing can include gently aspirating the droplet from the surface of the device and adding a 10 μl droplet of PBS to wash the surface. In some embodiments, target cells are captured by the CNT devices without pre-labelling, pre-fixation, or any other processing steps. In some embodiments, before spiking, it is possible to count the cells using hemocytometer and an optical microscope. After completing the process of capturing cells and washing the device, the cells should be stained with proper biomarkers in order to count them and confirm their viability under fluorescent microscope. In some embodiments, DAPI is used to stain nuclei. In some embodiments, cytokeratin is used to count cancer cells.

In some embodiments, the sample is a bodily fluid, such as saliva or urine sample. Processing these samples typically do not require removal of RBCs. However, in the event that RBCs are present in the saliva or urine (e.g., due to disease or injury), the RBCs can be removed via the lysis protocol described above. In some embodiments, instead of RBCs other cells that have a higher settling rate than the target cells may be removed. For example, cells with higher density may have higher settling rate.

The CNT system and method of the present disclosure can be utilized in accordance with a variety of applications. For example, it can be used for multi-market based captures, spiked cancer cells in studies, adherence capture based on nanosurfaces, classification of biomarker based on electrical signals, xenograph mice models, minimally invasive diagnosis based on blood tests, diagnosis of infectious diseases, and cell culture studies.

The devices and methods of the present disclosure are described in the following examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the embodiments of the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### EXAMPLES

FIG. 1C illustrates the fabrication process, which is described above. Four generations of devices consisting of 60-element, 76-element, and 240-element arrays on silicon wafers and 76 element arrays on glass wafers have been fabricated. Using an RBC lysis protocol presented herein for isolation of CTCs, one can process 8.5 ml of blood and isolate CTCs using the carbon nanotube chip and methods of preferential adherence as presented herein.

FIG. 1D shows the scanning electron micrograph (SEM) of single-walled carbon nanotubes. HiPCo carbon nanotubes about 1 nm in diameter and 1 μm in length were used. The nanotubes are transferred to the glass surface using a vacuum filtration process. The carbon nanotubes as seen in both SEM (FIG. 1D) and AFM (FIG. 1E) show random arrangement. FIG. 2C presents the entire wafer consisting of the 76-element array. The inset in this figure shows one embodiment of a single device that is 3 mm×3 mm. A single blood droplet is shown on the second inset. All the four sides of the device have a 30 μm thick SU8 layer that enables droplet localization due to hydrophobicity. FIG. 1H presents a Raman spectra of carbon nanotube films that indicate an RBM mode (275 cm⁻¹), a small D band (1336 cm⁻¹), a large G band (1591 cm 1) and pronounced 2D band or G′ band (2656 cm⁻¹). The large G/D band ratios indicate high-quality carbon nanotubes.

Tracking Single Cells Using a Carbon Nanotube (CTN) Chip

Target cells in a blood sample start to settle immediately due to gravity, and density gradients result in cells (e.g., RBCs) coming in contact and interacting with the bare nanotube substrate surface along with CTCs. The initial observations on the optical microscope of cancer cell spiked blood sample droplets showed that the spiked cancer cells and RBCs, as a part of the settling process, tend to go to the bottom of the device compared to WBCs, which do not settle in the same manner as RBCs. RBC's propensity to settle more quickly than other cell types is due to its iron content, which leads to its comparatively greater density.

To track individual cells, a TNBC cell line (MDA-MB-231; EpCAM-) that was transduced by lentivirus to actively express a green fluorescent protein (GFP) marker was used. Typically, CTC technologies such as CELLSEARCH® use Epithelial Cell Adhesion Molecule (EpCAM) to distinguish CTCs from hematological cells. CTCs are highly heterogeneous and actively change their shape, morphology, and even downregulate EpCAM during epithelial-mesenchymal transition (EMT). Thus, EpCAM-based methods fail to capture or otherwise detect CTCs and, therefore, are inadequate for clinical decision making.

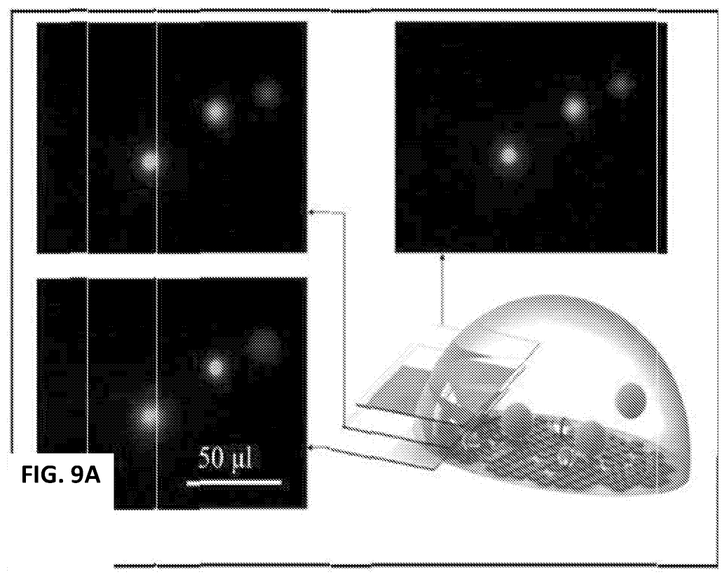

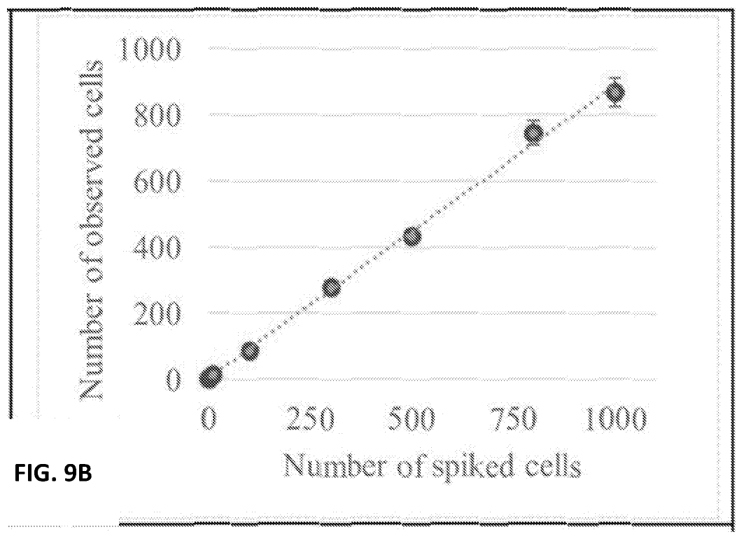

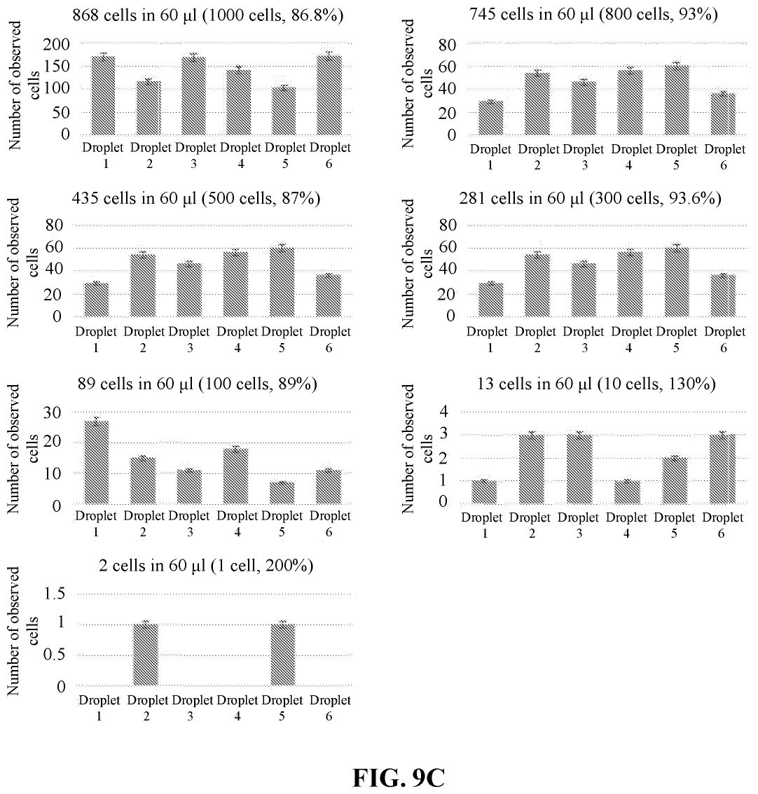

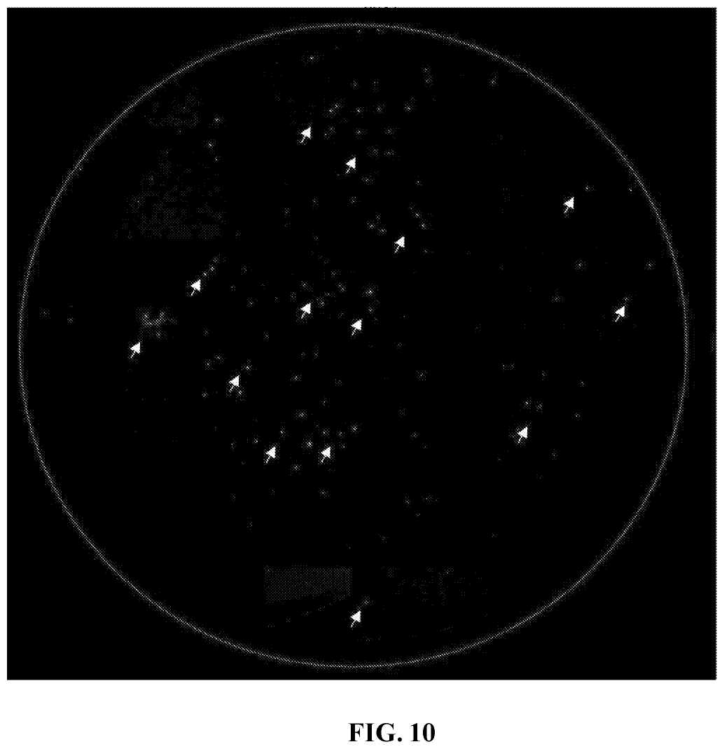

The GFP transduced triple-negative breast cancer cells were spiked into blood samples and observed under a fluorescent microscope. For these experiments, blood was diluted to 10%, which enabled tracking of the GFP+ cells in the droplet. FIG. 9A presents the fluorescent image of the GFP tracked cells at a different depth of focus. FIG. 9B shows the number of spiked cells compared to GFP+ cells observed in the samples. FIG. 9C presents the spiked cell counts in blood for 1, 10, 100, and 1000 spiked GFP+ cells in the blood. 87% to 100% capture of the GFP cells was observed. Slight errors in cell counts are a result of counting the cells using a hemocytometer. FIG. 10 is an image of a droplet with MDA-MB-231-GFP cells marked by arrows.

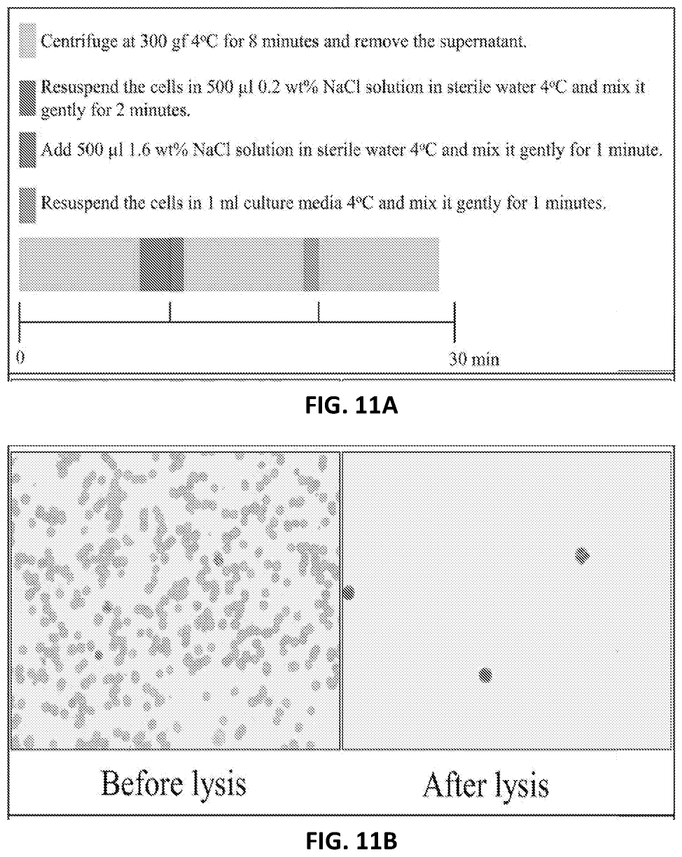

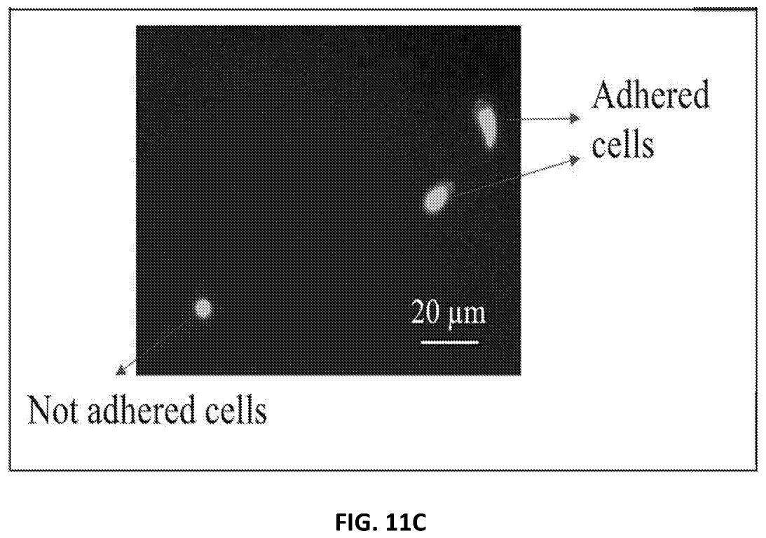

In the spiking experiments, it was observed that when a droplet of blood was placed on the nanotube device surface, the cancer cells and RBCs went to the bottom of the sample well (i.e., where the nanotubes are position) as a part of the settling process. The RBCs covered most of the nanotube surface, which is not desirable when using a preferential cell adherence strategy to capture or detect relatively rare CTCs. Exposing the cells to the nanotube surface is desirable because it enables cellular anchoring to the nanotube matrix. In many mechanobiology studies, microfabricated topographic features with specific dimensions mimic the architecture and orientation of the extracellular matrix (ECM) in vitro. The nanotube surface enables topographic anisotropy for cellular attachment due to the collection of nanometer scale tubes on the surface. For CTC isolation based on such topographic features, RBC lysis is necessary as this enables more exposure of the target cells to the nanotube surface. The disclosures presented herein have shown that RBC lysis enables capture of viable, high quality CTCs on non-functionalized carbon nanotube surfaces. Further, without RBC lysis, the efficiency of CTC capture, attachment on nanotube surface, and propagating cultures will be significantly reduced. FIG. 11A illustrates the RBC lysis protocol. FIG. 11B is an optical image of control blood (from a healthy volunteer) before and after lysis. WBCs, but not RBCs, are observed after the lysis procedure. FIG. 11C compares the cells that are attached to the nanotube surface to non-adhered cells using the RBC lysis protocol.

Preferential Adherence of Spiked Cancer Cells on aCNT Chip

Preferential adherence refers to the concept that CTCs preferentially attach to nanotube surfaces, but other blood components, including WBCs, do not. For example, nano-roughened glass surface adhesion-based capture of CTCs with heterogeneous expression and metastatic characteristics has been reported with capture yields of >80% for both EpCAM+ (MCF-7, SUM-149, A549) and EpCAM-(MDA-MB-231) cancer cell lines spiked in blood samples. This effect on carbon nanotubes has been demonstrated in the presently described microarrays, and capture efficiencies are more substantial with very high purity (5-log depletion), which is partially due to RBC lysis.

To determine if spiked cancer cells would survive the RBC lysis process, GFP positive, EpCAM-MDA-MB-231 breast cancer cells were spiked into mouse blood, and the lysis protocol was used. Five samples containing 1, 10, 100, 500, and 1000 MDA-MB-231 cells were spiked into 10 μl blood from wild type mice in 5 different 1.5 micro-centrifuge tubes. After each sample was lysed, the cells were resuspended in culture medium and divided onto six CNT chips (10 μl each). They were kept inside a sterile culture dish containing PBS to stop droplets from being dried in a 5% CO₂ incubator at 37° C. After 48 hours, samples were taken out from the incubator, and the droplet was removed and transferred into the second device to count the number of non-adhered cells from the second device. The first device was then washed with PBS, and both the primary and secondary devices were examined under a fluorescent microscope to count the cells on each device. The number of cells on the primary device was labeled “Adhered” while the ones on the secondary device were labeled “Not Adhered.”

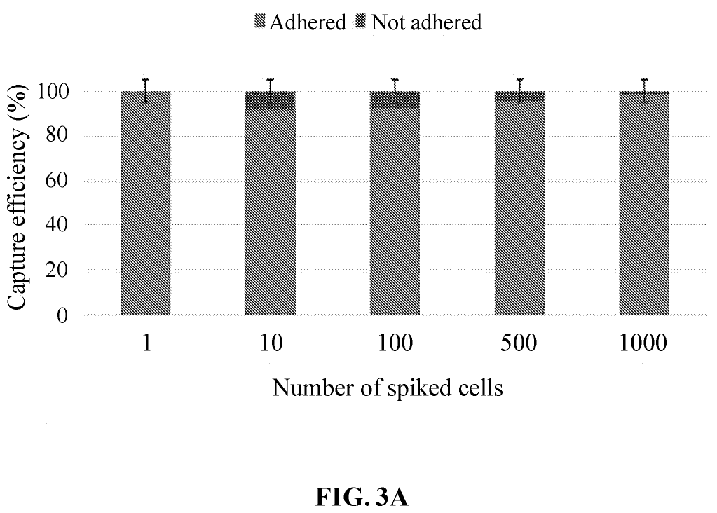

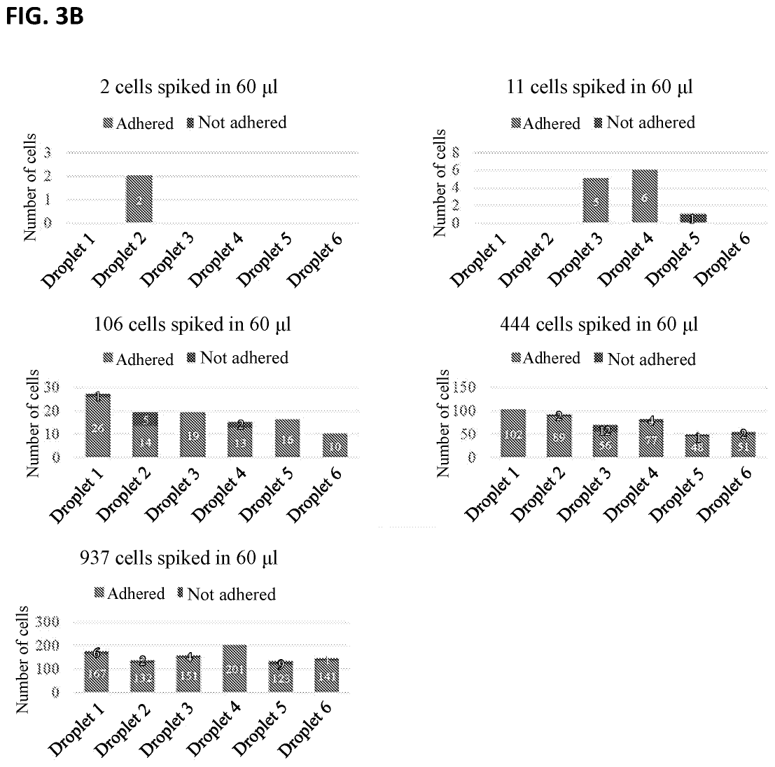

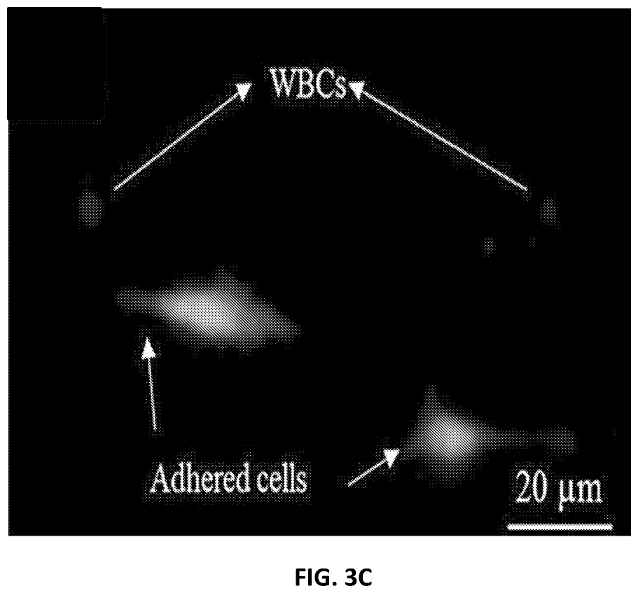

FIG. 3A shows that 87-100% of the target cells adhered to the CNTs on the primary device at all spiked concentrations. By using two devices from the array, both adhered and non-adhered cells were captured or all the spiked cells were tracked. FIG. 3B presents the number of cells counted in each droplet across all spike concentrations. The presently described methods comprise a new way to enumerate cells using droplets, having a standard volume, and a standard number of devices on an array. Since the volumes are quite small, one can ensure highly accurate counts. The standard 6 droplets can also be used for staining cells with 6 different markers, enabling multiple marker analysis of captured cells.

FIG. 3C presents the fluorescence image of the captured cells after RBC lysis and preferential attachment in spiked blood experiments. The difference in MDA-MB-231-GFP-Luc cells that are attached versus WBCs (DAPI only) on the same surface is observed. The cells that attached changed shape and morphology looking elongated/mechanically stretched as presented in the fluorescent images. The stages that characterize the process of static in vitro cell adhesion include the attachment of the cell body to the nanotube matrix, flattening and spreading, and the organization of the actin skeleton network with the formation of focal adhesion between the cell and the nanotubes. The deformation, mechanical stretching, and flattening are observed in FIG. 3C. However, to understand more about the cell adhesion, electron microscopy studies were conducted. Table 1 shows the number of captured cancer cells in spiked blood, the number of WBCs, and the log10 depletion of WBCs. An almost 4-log depletion in these small volumes was obtained, which demonstrates ahigh level of purity.

TABLE 1
Number of CTCs captured from spiked and patient blood.

| | Number of | Number of | | |
|---|---|---|---|---|
| | captured | captured | % WBC | Log₁₀ |
| Sample | CTCs | WBCs | contamination | Depletion |
| 1000 cells spiked | 937 | 12 | 0.016% | 3.79 |
| in 10 μl mice | | | | |
| blood | | | | |
| 500 cells spiked | 444 | 14 | 0.018% | 3.72 |
| in 10 μl mice | | | | |
| blood | | | | |
| 100 cells spiked | 106 | 21 | 0.028% | 3.55 |
| in 10 μl mice | | | | |
| blood | | | | |
| 10 cells spiked | 12 | 9 | 0.012% | 3.92 |
| in 10 μl mice | | | | |
| blood | | | | |
| 1 cell spiked | 2 | 16 | 0.021% | 3.67 |
| in 10 μl mice | | | | |
| blood | | | | |
| Patient 1 (8.5 ml) | 8 | 31 | 4.86 × 10⁻⁵% | 6.31 |
| Patient 2 (4 ml) | 39 | 637 | 2.12 × 10⁻⁵% | 4.67 |
| Patient 3 (4 ml) | 21 | 479 | 1.59 × 10⁻⁵% | 4.79 |
| Patient 4 (8.5 ml) | 238 | 277 | 4.34 × 10⁻⁶% | 5.36 |
| Patient 5 (8.5 ml) | 27 | 549 | 8.61 × 10⁻⁶% | 5.06 |
| Patient 6 (4 ml) | 4 | 151 | 5.03 × 10⁻⁶% | 5.29 |
| Patient 7 (8.5 ml) | 9 | 771 | 1.29 × 10⁻⁵% | 4.91 |
| Healthy control 1 | 0 | 643 | 1.008 × 10⁻⁵% | 4.99 |
| (8.5 ml) | | | | |
| Healthy control 2 | 0 | 652 | 1.022 × 10⁻⁵% | 4.99 |
| (8.5 ml) | | | | |

For calculation of WBC contamination and log depletion, a median of 7,500 WBCs per micro-liter was used. WBCs can be between 4,000 to 11,000 per micro-liter in healthy blood.

Electron Microscopy of Single-Cell Adhesion on Carbon Nanotube Surfaces

Electron microscopy studies of attached single cells were conducted to investigate how cancer cells attach to a nanotube surface. FIG. 4A is a scanning electron microscope (SEM) image of an attached SKBR3 cell that was incubated for 48 hours on a CNT device. The striking image shows that cancer cells change morphology and spread on the nanotube surface causing strong focal adhesion. The filaments from the main body of the cell extend to the nanotube surface. Many such filaments are observed to attach to the individual nanotubes/bundles directly. The average diameter of these filaments is about 150 nm to 200 nm and cannot be seen under an optical microscope. The exposure of some of these filaments under an SEM demonstrates that thousands of such filaments bond to the nanotube matrix. Integrin receptors are vital in static in vitro cell adhesion and spreading. Specific integrin binding provides a mechanical linkage between the intracellular actin cytoskeleton and nanotube matrix. The electron microscopy studies confirm that tumor derived epithelial cells attach firmly to the CNT surface, including individual CNT bundles, and exhibit active dynamics.

FIG. 4B correlates the time of adherence and the captured number of cells. Three different samples containing 50 MDA-MB-231-GFB-Luc cells were mixed with 10 μl wild mice blood inside a 1.5 ml tube and lysed. 10₁11 droplets of each of the three sample was placed on three separate CNT devices as a and incubated for 12, 24, and 48 hours. The droplets were removed from the CNT devices and then placed on new CNT devices for another 72 hours to observe if any of the non-attached cells could be attached to the surfaces of the new CNT devices. For the first sample, which was incubated for 12 hours, <30% of the cells attached to the CNT device at the first step. An additional 50% attached after 72 hours. However, when the initial incubation time was increased to 48 hours, more than 90% of the cells attached, indicating an increase in adhesion strength with time. The strength of adhesion of the cell to the nanotube matrix is proportional to the length of incubation time. The advantage of a small adherence time is that only 7 WBCs (less than 0.005%) adhered to first CNT device. For the second sample, 42 out of 52 cancer cells (80%) adhered to CNT surface after 24 hours. 4 cells adhered on the secondary device after 48 hours and 6 cells (11%) were not captured. For the third sample, 45 of 49 cells (92%) adhered to CNT film. The unadhered cells also did not adhere to the second device. 21 and 24 WBCs (0.02%) in the second and third samples, respectively, adhered to a CNT surface. From these experiments, it was concluded that 48 hours is the optimum incubation time to have the highest capture efficiency with negligible WBC capture.

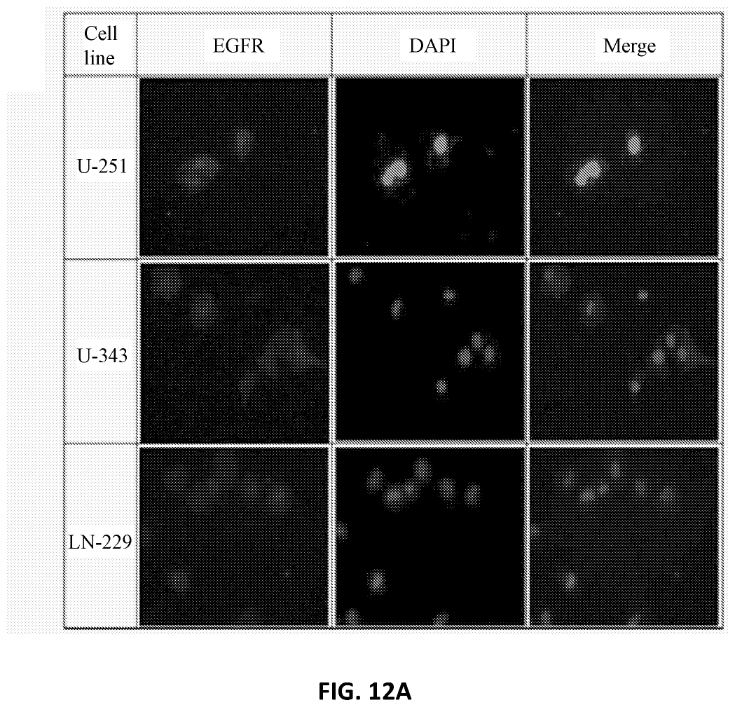

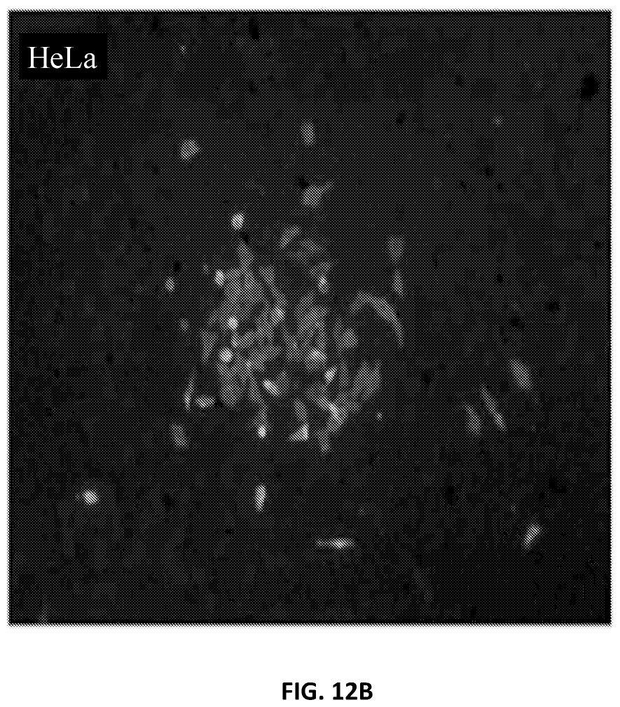

While all the experiments used GFP+ MDA-MB-231 triple negative cells, this technique is not limited to specific cancer types, and therefore could potentially capture any type of epithelial cancer (e.g., breast, colon, lung, and prostate) cell using the methods presented herein. Further, the novel method disclosed herein is the only method that track both adherent and non-adherent cells on the same chip. The method, therefore, effectively tracks all cells in a sample, a task that is of high value in CTC capture especially in early-stage cancers when cell (e.g., CTC) numbers may be meager. Other epithelial cancer cell lines including HeLa (Cervix), U-251 (Glioblastoma), MCF7 (Breast), LN-291 (brain) were also tested using this method, and the yields of adherence were also more than 90%. FIG. 12A is a representative image of the brain cancer cells that were stained for DAPI and EGFR. FIG. 12B shows adhered HeLa cells stained with CD59 after adherence to CNT film. With a suitable functionalization protocol, one can also capture non-epithelial cells such as lymphoma and sarcoma cells.

Preferential Adherence Using Collagen Adhesion Matrix on aCTN Chip

The presently disclosed cancer cell attachment strategy on carbon nanotubes was compared with that of collagen adhesion matrix (CAM) scaffolding for the capture of CTCs. The capture of CTCs using a CAM strategy (Vita assay) is a unique strategy and is a method of adherence. The ability of a tumor cell to invade collagenous matrices is one of the hallmarks of metastasis. In the past, it was hypothesized that populations of CTCs that adhere and invade collagenous matrices would be invasive and exhibit the natural tendency to undergo metastasis in vivo.

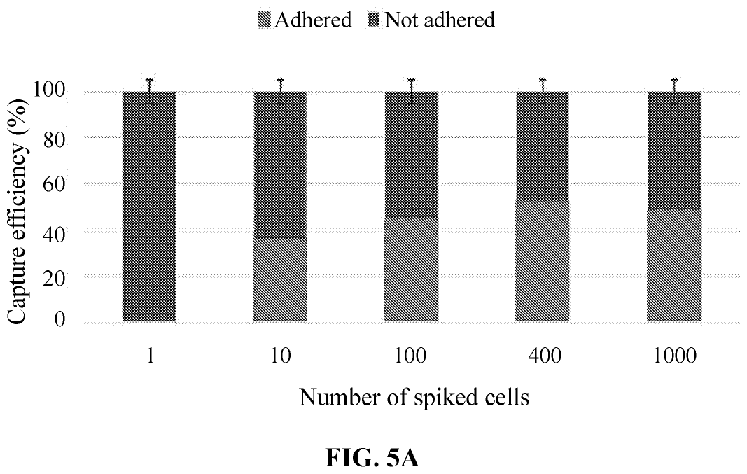

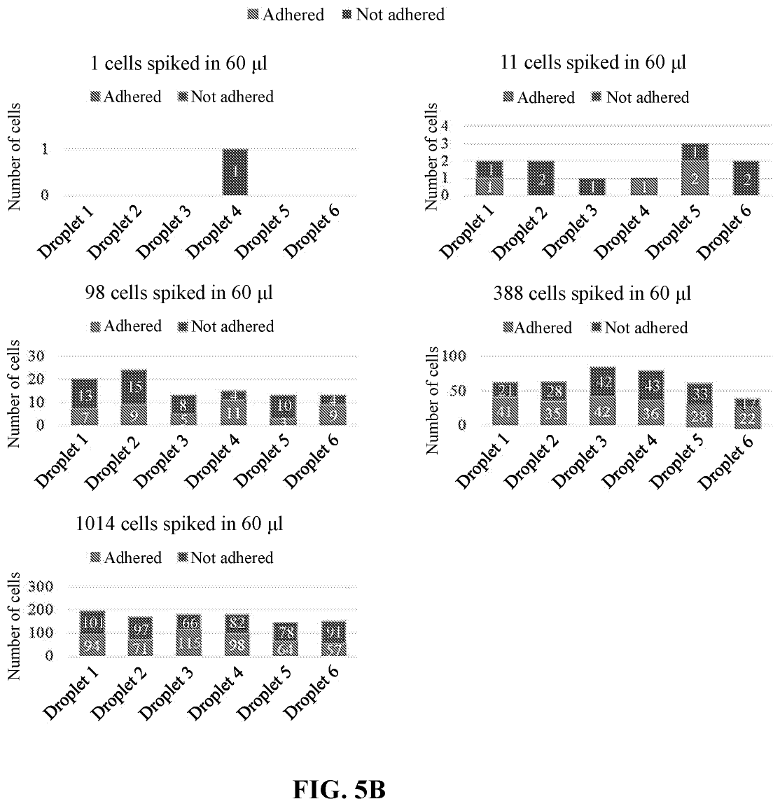

FIGS. 5A and 5B presents the overall capture efficiency and the number of cells (both adhered and non-adhered) for each device for the CAM strategy. The adherence efficiency of the CAM strategy is observed to be only 50%, which is consistent with what has been reported previously (J. Lu et al., Isolation of circulating epithelial and tumor progenitor cells with an invasive phenotype from breast cancer patients, Int. J. Cancer, 2010, 126, 669-683). 50% of cells did not adhere on the primary CAM device even after 48 hours incubation, although all the cells (i.e, both adhered and non-adhered cells) were tracked by counting the number of cells in the secondary device. These experiments were repeated three times with similar capture rate values. It is possible that CAM devices need more time for cells to digest the collagen, although it should be noted that some of the cells may not attach to the CAM. A study based on CAM coated tubes in Stage I-III breast cancer (Vita-assay) only detected CTCs in 28/54 patients (i.e., 52% sensitivity). The CAM strategy needs optimization regarding collagen type, the deposition technique, and concentration for the CNT chip. This method has a lower yield compared to bare single wall CNT film under the same conditions for preferential adherence using the microarrays presently disclosed.

Clinical Studies: Capture of CTCs from Breast Cancer Patients Using a CNT Chip

The ability of the CNT chip to isolate CTCs with high purity from breast cancer patients was investigated. De-identified 8.5 ml blood samples were obtained from the University of Louisville under IRB (IRB#18.0828). De-identified 4 ml blood samples were also obtained from UMASS tissue bank to determine the numbers of CTCs captured from samples obtained from different sources and having different volumes using the CNT chip.

FIG. 6 is a representative image of CTC identification and capture in a breast cancer patient compared to no capture of CTCs in a healthy control. CTCs were identified and scored as cells that were clearly visible and possessing cancerous cellular morphology under an optical microscope, positive for nuclear stain DAPI, positive for CK (8/18), and negative for CD45. Because CTCs are often no larger than WBCs and because WBCs have a nucleus, it is important to distinguish between CTCs and WBCs for scoring purposes. Lymphocyte common antigen CD45 is expressed in all leukocytes, and, therefore, WBCs are identified as cells that are clearly visible under an optical microscope, positive for lymphocyte antigen CD45, negative for CK 8/18, and positive for nuclear stain DAPI. CTCs do not have to be any larger than leukocytes. Size selective techniques that capture CTCs based on the assumption that epithelial CTCs are much larger than leukocytes may not capture all CTCs. The CNT chip enables the capture of CTCs without size bias or relying on antigen expression, thereby capturing all (or nearly all) CTCs in a sample.

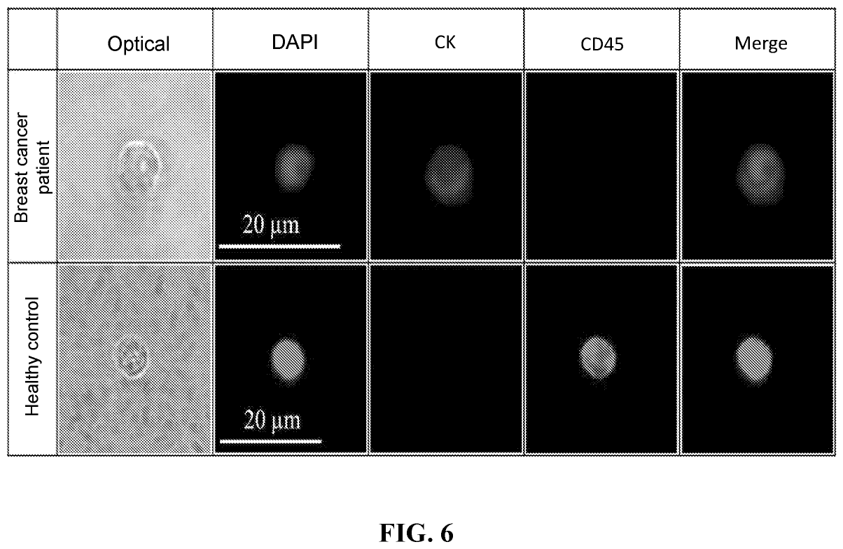

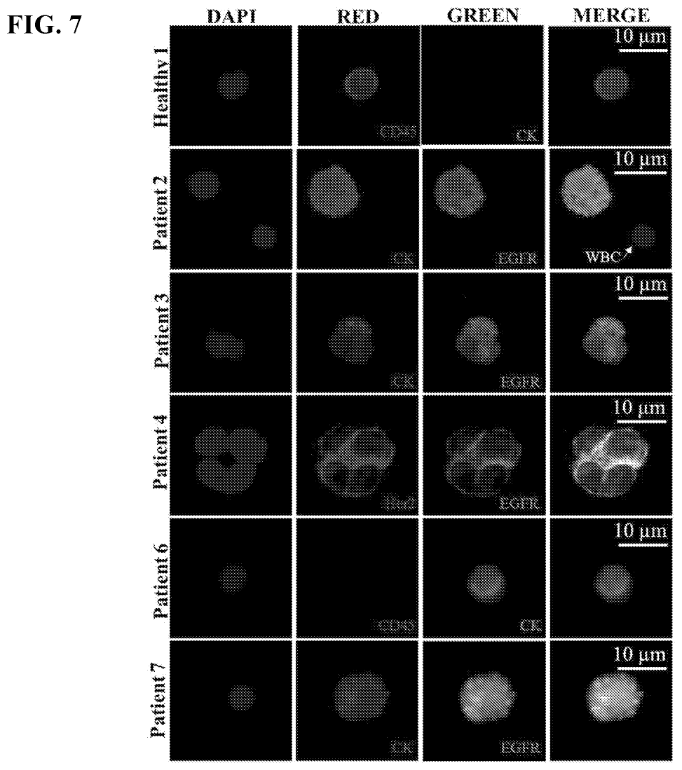

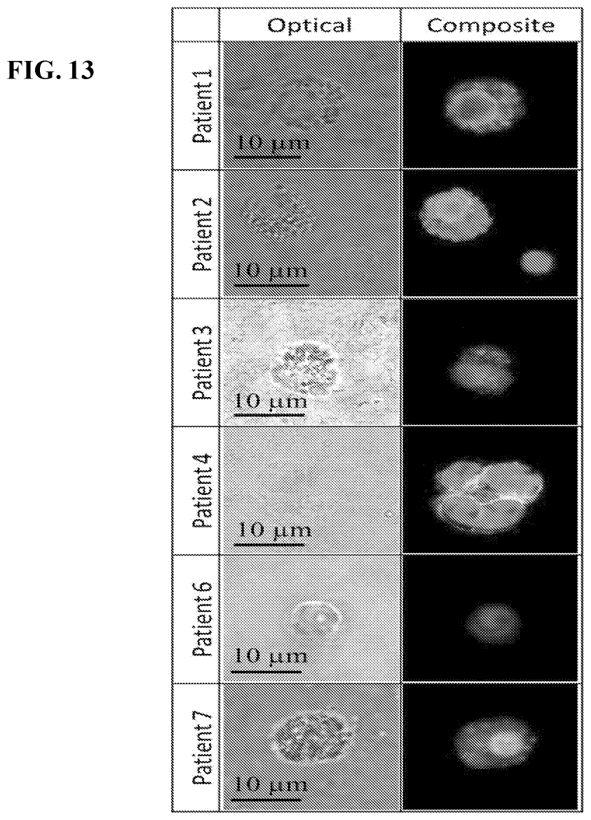

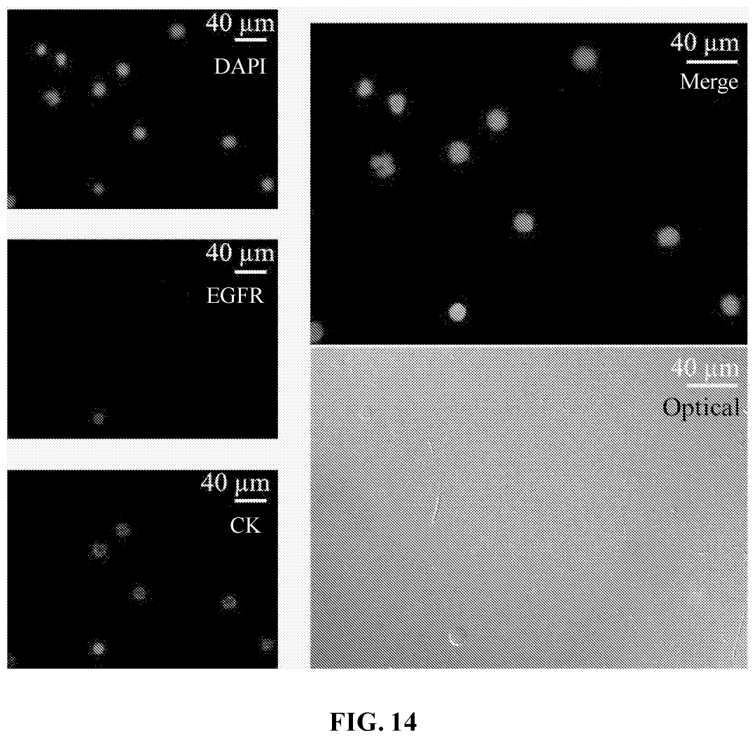

FIG. 7 is the representative CTCs captured in patients using multiple antigenic markers in 4 ml and 8.5 ml blood. CTCs were captured in all patient samples (n=7/7), suggesting 100% sensitivity. Healthy controls (n=2/2) showed no presence of CTCs in blood. All optical images and merged images from patients are presented in FIG. 13. CTCs of different phenotypes were captured based on CK8/18+, Her2+, and EGFR+expression. Both healthy control 1 and 2 were CK8/18−, CD45+, and DAPI+. CTCs from patients were CK(8/18)+/Her2+/EGFR+, CD45−, and DAPI+.

Table 2 presents the tumor nodes metastasis (TNM) staging and the number of CTCs and heterogeneous CTCs captured using a CNT chip.

TABLE 2
Patient characteristics, TNM staging, CTC numbers, and stable/progressive disease

| | TNM staging | # CTC | #CTC | | #Heterogeneous | |
|---|---|---|---|---|---|---|
| Patient | and Source | captured | per ml | | CTCs | Notes |
| Patient 1 | PT4N2M1; | 8 in 8.5 | N/A | | 8 based on | Lysis used more |
| | treatment naïve; | ml blood | | | Her2/EGFR | than once. A |
| | Stage 4; UofL; | | | | | criterion cannot |
| | | | | | | be established |
| Patient 2 | PT1CN0; Stage | 39 in 4 | 9.75 | CTC/ml | 36 CK+ and 3 | Stable disease; |
| | 1B; UMASS; | ml blood; | | | EGFR+ | lymph node |
| | | | | | | invasion |
| Patient 3 | PT2N0M0; | 21 in 4 | 5.25 | CTC/ml | 19 CK+, 2 EGFR+ | Stable disease; |
| | Stage 2 | ml blood | | | | tumor >20 mm |
| | UMASS; | | | | | |
| Patient 4 | PT1BN1M1; | 238 in 8.5 | 28 | CTC/ml | Her2+ and EGFR+ | 2 CTC clusters; |
| | Stage 4; UofL; | ml blood | | | | progressive |
| | treatment naïve; | | | | | disease |
| Patient 5 | PT1N1M0; | 27 in 8.5 | 3 | CTC/ml | 3 EGFR+ and 26 | Stable disease |
| | treatment naïve; | ml blood | | | CK+ | |
| | Stage 2A; UofL | | | | | |
| Patient 6 | PT2N2A; Stage | 4 CTCs | 1 | CTC/ml | only CK+ | Stable disease |
| | 3A; UMASS | in 4 ml | | | | |
| | | blood | | | | |
| Patient 7 | PT4BN1M1; | 9 CTCs | 1 | CTC/ml | 1 EGFR; 8 CK+ | Metastasis to |
| | treated with | in 8.5 ml | | | | bone and lung; |
| | radiation; UofL | blood | | | | CTCs still exist |
| | | | | | | after radiation |
| | | | | | | therapy |

Patient 1 (stage 4) was an outlier as the lysis procedure was not successful initially (due to platelet aggregation) and was performed more than once. However, 8 CTCs expressing Her2 and EGFR were still captured (FIG.13). For all but the first patient, a whole blood stabilization agent (tirofiban; 0.5 μg/ml) was added before shipping at 4° C. For all but the firstpatient, the protocol was uniform across all the samples. As Table 4 suggests, anywhere from 8-238 CTCs per sample were captured in 4 ml and 8.5 ml blood. CTCs were captured in patients that were lymph node positive and negative. In general, using TNM staging and number of CTCs counted, it was inferred that patients who were between stages 1-3 (patient 2, patient 3, patient 5, and patient 6) had a lower number of CTCs (4-39 CTCs in 4 ml and 8.5 ml blood or 0.5 to 10 CTCs per ml) than patients with stage 4 cancer. For example, patient 4 had stage 4 breast cancer and an elevated level of CTCs (238 in 8.5 ml blood or 28 CTCs per ml) before treatment. There is an apparent spread in CTC counts between early stage (stage 1-3) and advanced disease (stage 4) using the CTN chip. The CTCs were positive for both Her2 and EGFR suggesting aggressive disease. Two Her2+/EFGR+clusters were also observed in patient 4. Finally, in patient 7, blood was only obtained after radiation therapy (although the patient was chemo naive). Surprisingly, only 9 CTCs were captured in 8.5 ml blood (1 CTC/ml), indicating that the number of CTCs may have receded in this stage 4 patient after radiation. Comparing both stage 4 patients, patient 7 (radiation therapy) to patient 4 (treatment naive), the CNT chip predicts treatment response based on CTC enumeration.

CTC Purity in Patient Samples

It is important to capture CTCs of high purity to enable further genomic characterization. Purity describes the ability of the device to capture CTCs in a sample comprising contaminating leukocytes. Purity is a metric that can be measured from clinical samples. Using a CTN chip, a log10-depletion for each patient based on the number of WBCs captured was established. The log depletion formula used to assess CTC purity follows:

$$Depletion = {log}_{10} - \frac{\# WBCs initial}{\# WBCs final}$$

Using this formula, the log-depletion of WBCs in each of the patient and control samples was assessed. Table 2 shows 4- to 5-log depletion of WBCs. Patient 1 is an outlier due to the lysis procedure being performed more than once as explained above. Both healthy controls exhibited the same log depletion, indicating high controllability and uniformity of the process. The range of log depletion was between 4.6 to 5.3, which indicates that this number could be used as a calibration marker for process control in routine clinical practice. Narrowing this distribution enables comparison across multiple cancer types.

Capture of CTCs of Various Phenotypes from Breast Cancer Patients Using a CTN Chip:

One of the objectives of the patient study was to investigate the presence of single CTCs of various phenotypes (Her2+/EGFR+ CTC subclones). The EGFR family of receptors is comprised of EGFR (ErbB-1, HER1 in humans), HER2 (ErbB-2), HER3 (ErbB-3), and HER4 (ErbB-4). There is a strong interest recently in EGFR and HER2 because of their overexpression in breast carcinomas. EGFR (HER1) signaling induces epithelial to mesenchymal transition (EMT) through different pathways that results in tumor progression and metastasis. In the analysis of all patients, apart from CK(8/18)+ cells, 2-3 cells that were strongly EGFR positive in stage 1-3 cancer were captured. This indicates that CTCs of various phenotypes exist in patients even in early-stage cancers. However, advanced stage cancer patients (e.g., patient 4) showed both Her2 and EGFR positive CTCs with large numbers of CTCs (238). This indicates that the combination of CTC numbers and the heterogeneity of CTCs determine the aggressiveness of breast cancer. FIG. 14 shows a single CTC at the bottom of the image exhibiting multiple phenotypes (both EGFR and CK(8/18 positive)), while the majority of CTCs were only positive for CK8/18. This suggests a small clone exists among primary tumor CTCs that have metastatic or EMT phenotype. The question is whether such small subclones (expressing EGFR family of receptors) resist chemotherapy and result in progressive disease and metastasis few years after treatment. The presence of CTCs in circulation may be linked negatively to the survival of a patient. Brain metastatic breast cancer (BMBC) can express both Her2 and EGFR, indicating that such populations are at high risk for metastatic disease. The high CTC numbers and the presence of both Her2 and EGFR pathological features can trigger EMT, metastasis and determine future survival. The CNT chip thus enables capture of these small subclones and better insights of disease progression.

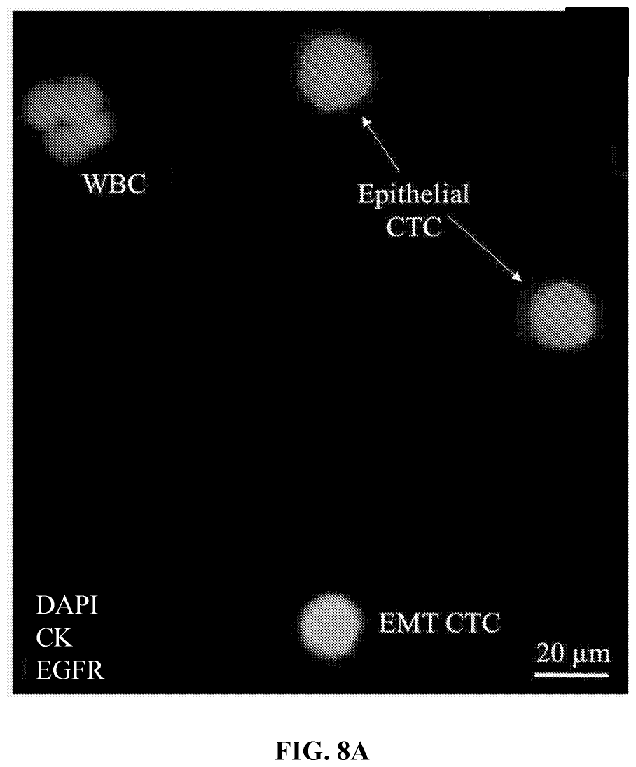

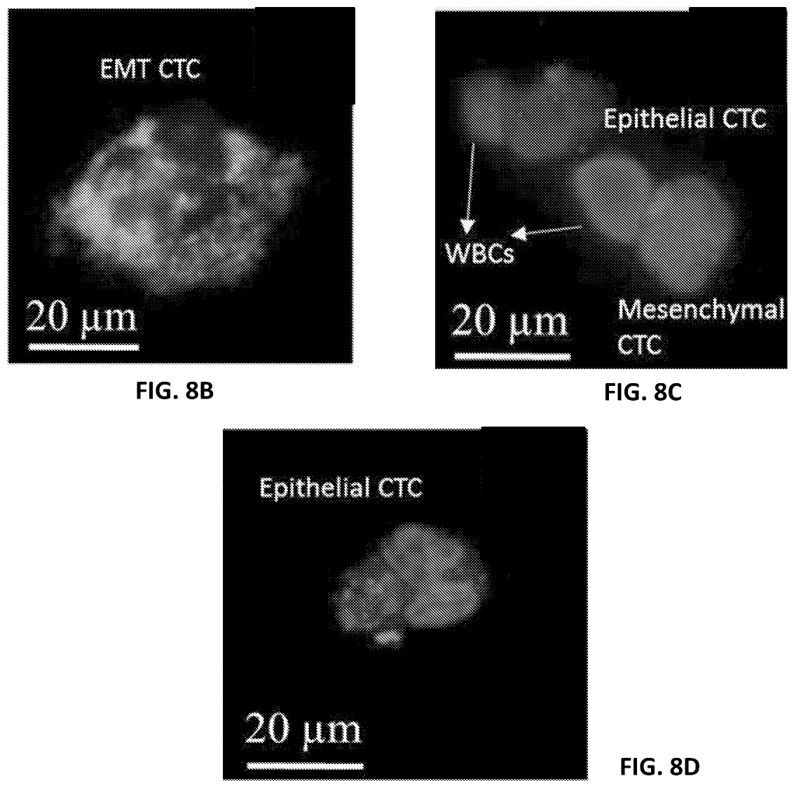

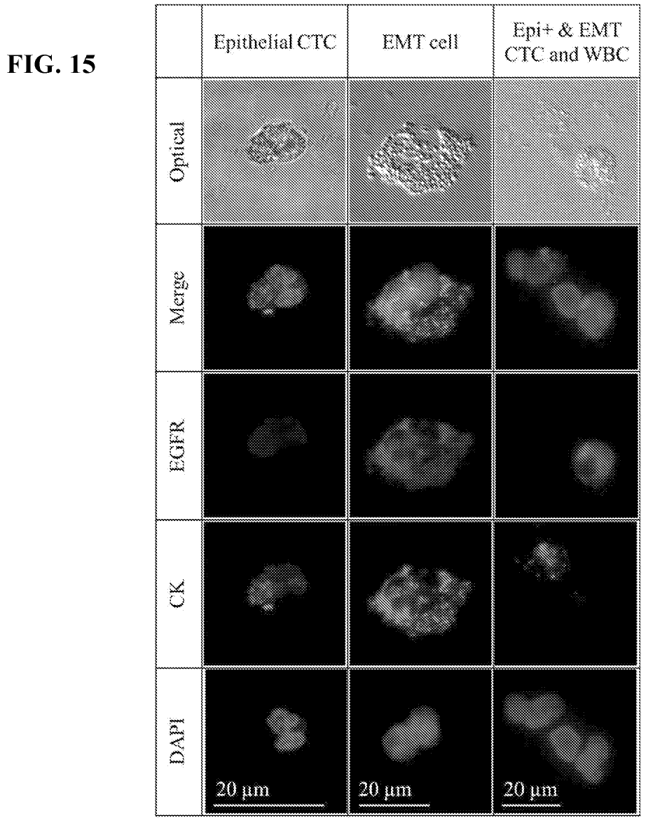

To distinguish between CTCs of different phenotypes, whether different types of CTCs could exist in same patient sample was investigated. FIG. 8 provides dynamic views of epithelial and mesenchymal states of CTCs captured from patient 5. In FIG. 8A, WBC (DAPI only), epithelial CTCs (positive only for CK8/18), and EMT related CTCs (CK8/18 and EGFR) are present in the sample. FIGS. 8B-8D show the different CTCs from the same sample. In FIG. 8B, the spindle cell with both EGFR and CK8/18 expression indicates activation of the EMT process. CTCs often change morphology upon EMT activation, and the presence of EGFR and the morphology of the CTC can be a positive confirmation. In FIGS. 8C and 8D, the presence of both epithelial and mesenchymal CTCs is observed. Epithelial CTCs were only positive for CK8/18, but the more aggressive mesenchymal state was also strongly positive for EGFR and CK8/18 expression was absent (FIG. 8C). Overall, three EGFR+ CTCs and 26 CK8/18+CTCs were observed in patient 5, indicating that the CNT chip can track CTCs of various phenotypes at the single cell level. FIG. 15 shows the difference between epithelial+, mesenchymal+, and EMT+ CTCs. Analysis of phenotype heterogeneity focused mainly on three markers, CK (8/18), Her2, and EGFR, can inform decision making regarding available treatment options are available. Overall, the CTN chip enabled a high level of success in analyzing CTCs, CTC enumeration, the capture of heterogeneous CTCs, and the ability to distinguish between epithelial, mesenchymal, and EMT related CTCs in breast cancer patients.

Comparison of CNT Chip with Existing CTC Capture Techniques

Table 4 compares some of the existing CTC capture methods with that of the CNT chip of the present disclosure. The CELLSEARCH® technique, based on EpCAM antigen-dependent capture and immunomagnetic enrichment, was the first to arrive in the market. A decade of research on CTC capture based on CELLSEARCH® has only yielded modest results. In the recent German SUCCESS study using the CELLSEARCH® system and involving 2026 breast cancer patients before chemotherapy and following surgical removal of primary tumor, CTCs were detected in only 21.5% of patients (n=435 of 2026). A study comparing CELLSEARCH® and ISET (filtration system) for circulating tumor cell detection in patients with metastatic carcinomas yielded consistent results. CTCs were detected in only 55% (11 out of 20) of the patients with breast cancer, 60% (12 out of 20) of the patients with prostate cancer, and only 20% (4 out of 20) of lung cancer patients. Discrepancies between the techniques regarding the number of CTCs detected were observed.

Microfluidic technologies such as CTC-Chip, the Herringbone chip, and the CTC-iChip are fluidic platforms. The CTC-chip with micro-posts is challenging to manufacture and functionalize its micro-posts' surface, and no CTC clusters were captured using this device (M. M. Ferreira et al., Circulating tumor cell technologies, Mol. Oncol., 2016, 10:374-394). The Herringbone chip, a planar device from which collecting CTCs is difficult, yielded only 2 clusters. The CTC iChip exhibits low WBC contamination and can capture CTCs antigen-dependently or independently. But an array with 20 μm gaps on the iChip cannot capture CTC clusters and thus is reduced to size-dependent capture. Therefore, newer devices with asymmetry and size-based separation are being developed. Most of the filtration and size-based techniques such as ScreenCell, MOFF, and ISET isolate CTCs. However, red blood cell (RBC) saturation and clogging are problems with these devices. Similarly, microfabricated filters also isolate CTCs. The problem with most size-based technologies is that the CTCs are highly deformable unless fixed chemically, and EMT related CTCs are often not retained.

Compared to these techniques, the CNT chip has distinct advantages. The CNT chip is a novel antigen-independent and size-independent capture technique based on the mechanobiology of tumor cells allowing attachment to nanotube surfaces. The preferential adherence strategy enables 5-log WBC depletion, which compares favorably to other CTC capture techniques. The capture yield is 100% for low levels of spiked triple-negative breast cancer cells (1, 10, 100), suggesting that the RBC lysis and preferential attachment of cancer cells to a nanotube surface is a highly effective strategy. The CNT chip was used successfully in capturing heterogenous CTCs present in 4 ml and 8.5 ml samples derived from patients having different stages of breast cancer. In a single microarray, the CNT chip demonstrated the correlation between advanced disease, high CTC numbers, CTC pathological features, and ability to track a single CTC with multiple phenotypes.

Materials and Methods

Cell Culture

The breast adenocarcinoma cell lines Luciferase/Green Fluorescent Protein (GFP) dual-labeled MDA-MB-231 was cultured in RPMI-1640 growth medium, MCF7 breast cancer cells were cultured in EMEM growth medium, SKBR-3 breast cancer cells were cultured in McCoy's 5a growth medium, cervical adenocarcinoma cell line HeLa was cultured in low glucose DMEM growth medium, and brain cancer cell lines U251, U-343, LN-229 were cultured in low glucose DMEM growth medium as per their suggested protocol by manufacturer. All media contain 10% Fetal Bovine Serum (FBS) and 1% penicillin-streptomycin. The cell lines were incubated at 37° C. 5% CO2. 0.25% EDTA-trypsin solution was used to resuspend cells.

Carbon Nanotube (CNT) Film Fabrication

Super pure small diameter Unidym™ HiPCO Single Wall Carbon Nanotubes (SWCNTs) were purchased from a commercial vendor. 100 μg of SWCNT powder was dispersed in 100 ml of isopropyl alcohol (IPA). After sonication for 24 hours, the dispersed SWCNTs were filtered on a 220-nm pore size 90 mm diameter mixed cellulose ester filter membrane purchased from Millipore using vacuum filtration. The vacuum filtration method self-regulates the creation of a CNT network, and it produces a film with evenly distributed CNTs. Next, the CNT film on the membrane was pressed onto a 4″ glass wafer with a thickness of 500 μm. Using an acetone bath, the filter membrane was removed, and a transparent CNT film (75 mm diameter) on the glass wafer remained.

Characterization of Carbon Nanotube Film

After transferring the CNT film to a glass wafer, multiple methods were utilized to characterize the CNT film. Raman spectroscopy measurements were performed using a Horiba XploRa Raman spectrometer in an ambient environment by a green laser (excitation laser line of 532 nm). A 100× objective lens was employed to focus the laser beam on the CNT film, and the measurements were conducted with 1200 g/mm grating, 1% neutral density (ND) filter and a 0.2 mW laser power to avoid damaging the samples. For calibration, the phonon mode from the silicon substrate at 520 cm⁻¹ was used. Atomic force microscopy (AFM) images were acquired using a NaioAFM (Nanosurf Inc) in tapping mode with a cantilever resonance frequency of ˜146 kHz. Scanning electron microscope images of the CNT film were obtained using a JEOL JSM-7000F instrument at 10 kV and under an ultra-high vacuum (10⁻⁵ Pa).

CNT Chip Micro-Array Fabrication:

The CNT chip was fabricated in the Cleanroom at Boston College. The 76-element array chip fabrication is detailed elsewhere.

Spiking Cancer Cells into Mice Blood:

Cells were grown to reach ˜80% confluence. Cells were then washed with PBS and detached from the culture dish using Gibco™ Trypsin-EDTA (Cat No. 25200056). Next, they were centrifuged and suspended in a specific culture media volume, and a hemocytometer was used to count the cells and calculate their concentration in each tube. In order to be able to track the cells in blood using the fluorescent microscope for counting and calculating the capture efficiency of the devices, MDA-MB-231 GFP cells were used for most of the spiking experiments. 10 μl of wild mice blood was mixed with 10 μl of culture media containing the required number of cells in 1.5 ml tube. After mixing the target cells in blood, red blood cells (RBCs) were lysed. After centrifugation, the supernatant containing blood serum and lysed RBCs was removed, and the pellet at the bottom of the tube was resuspended in culture media and transferred onto the CNT device surface.

Red Blood Cell (RBC) Lysis for Spiking Experiments:

Hypotonic NaCl solution was used for RBC lysis. The collected blood from the mouse model or the spiked cells in blood was centrifuged at 300 ×g at 4° C. for 8 minutes, and the supernatant was removed. The cells were resuspended in 500 μl of 0.2 wt. % NaCl solution in sterile water at 4° C. and mixed gently for 2 minutes. Then, 500 μl of 1.6 wt % NaCl solution in sterile water at 4° C. was added and mixed gently for 1 minute. The solution was centrifuged at 300 ×g at 4° C. for 8 minutes, and the supernatant was removed. The cells were resuspended in 1 ml culture media at 4° C. and centrifuged at 300 ×g (4° C.) for 8 minutes, and the supernatant was removed. Cells were resuspended in 60 μl culture media and 10 μl of the processed sample was transferred to six different chips.

Preferential Attachment Studies:

Several experiments were designed to determine the optimized time of CTC attachment to the nanotube surface. Three different samples containing 50 MDA-MB-231-GFB-Luc cells were mixed with 10 μl wild type mouse blood inside a 1.5 ml tube and lysed. Each of these three samples was placed on three separate CNT devices as a 10 μl droplet and incubated for 12, 24, and 48 hours. After this time, the removed droplet was then placed on another new CNT device surface and incubated until the combined time for both incubations reached 72 hours. The second step was carried out to determine if non-attached cells could be attached to a new CNT device surface.

Preferential Attachment Using Collagen Adhesion Matrix:

Collagen adhesion matrix (CAM) was deposited on the surface of CNTs, and cell spiking experiments in blood were conducted. During these tests, the surface of the sensor was covered with collagen to improve and speed up the adhesion of target cells to the surface, similar to metastatic invasion by digesting the collagen. Collagen from calf skin Type I (0.1% solution in 0.1 M acetic acid), aseptically processed, and suitable for cell culture was used. Based on the suggested protocol by the manufacturer, a collagen solution (Sigma Aldrich Cat. No. C8919) was used to coat devices with 6-10 μg/cm² with a 10 μl droplet. The CAM droplet on the device was kept at 4° C. overnight to allow the proteins to bond with CNTs. The excess droplet was removed from the coated surface the next day. The device was dried overnight and simultaneously allowed to sterilize through exposure to UV light in a sterile biosafety cabinet. The following day, before using the device, it was rinsed with PBS.

Five samples containing 1, 10, 100, 400, and 1000 cells were spiked in 10 μl wild mice blood in 5 different 1.5 ml micro-centrifuge tube. After each sample was lysed, the cells were resuspended in culture medium and divided between six CNT chips, with each chip receiving 10 μl of sample. Incubation conditions and time was the same as previous spiking experiments. The same counting strategy was utilized to count the cells that were adhered to the primary device and not adhered on the secondary devices based on CAM strategy.

Patient Samples:

De-identified blood samples were collected in BD-vacutainer sodium heparin blood tubes (green cap). The volume of collected blood at University of Louisville cancer center was 8.5 ml, and the volume of collected blood in UMASS-tissue and biobank was 4 ml. After collecting the blood, 0.5 μg tirofiban was added to each ml of the blood sample. The sample was kept in a 4° C. refrigerator inside a biohazard specimen transport bag before being used or shipped. During shipping, blood samples were preserved between 2-8° C. in nano cool boxes. Before processing each sample, a smeared blood sample on a glass slide was stained with Giemsa stain (Sigma Aldrich #GS500) for detailed inspection of the blood sample.

Patient Blood Sample Processing

Collected blood samples were transferred from their original tubes to 15 ml centrifuge tubes and centrifuged at 300 ×g for 5 minutes. The blood plasma was removed from the supernatant. Each cell pellet was resuspended in 12 ml of lysis buffer (G Bioscience #786650). After mixing for 3 minutes, the tube was centrifuged at 130 ×g for 5 minutes. The supernatant of the lysed sample was transferred to a waste tube. Using 1 ml of culture media, the pelleted lysed cells were resuspended and transferred to a 1.5 ml microcentrifuge tube. Cells were centrifuged at 130 ×g for 5 minutes and supernatant transferred to a waste bottle. The remaining cells were resuspended in 120 μl culture media and divided between 12 CNT devices (10 μl of sample per CNT device). The devices were incubated at 37° C. 5% CO2 in a petri dish containing PBS. After 48 hours, the droplets on the devices were removed, and the devices were washed once with PBS. The isolated cells on the device were then used for immunofluorescence studies.

Immunofluorescence Analysis:

Captured cells were fixed with 4% paraformaldehyde for 10 minutes. The sample was washed with PBS and then blocked with Image-iT™ FX Signal Enhancer and immunofluorescence locking buffer (cell signal #12411) for 1 hour each at room temperature. The sample was covered with a primary antibody diluted based on the manufacturer's suggested concentration and incubated at 4° C. overnight. The sample was washed with PBS 3 times. The secondary antibody was diluted to 1 μg/ml, and the sample was covered for 1 hour at room temperature in a dark container. The sample was stained with DAPI. The number of CTCs was 8-238 CTCs per 8.5 ml or 4 ml blood; 7/7 patients with stage 1c to stage 4 cancers had CTCs. The number of WBC was 5 log to 6 log depletion of WBCs in patients; 31-652 WBCs in 8.5 mL or 3.6 to 75 per mL.

All patents, patent applications, and published references cited herein are hereby incorporated by reference in their entirety. It should be emphasized that the above-described embodiments of the present disclosure are merely possible examples of implementations, merely set forth for a clear understanding of the principles of the disclosure. Many variations and modifications may be made to the above-described embodiment(s) without departing substantially from the spirit and principles of the disclosure. It can be appreciated that several of the above-disclosed and other features and functions, or alternatives thereof, may be desirably combined into many other different systems or applications. All such modifications and variations are intended to be included herein within the scope of this disclosure, as fall within the scope of the appended claims.

1. A method for capturing and detecting target cells in a sample, the method comprising:
removing red blood cells from a blood sample,
depositing the blood sample on a device comprising one or more carbon nanotubes arrays formed with carbon nanotubes, wherein surfaces of the carbon nanotubes are not functionalized; and
detecting target cells adhered to the carbon nanotubes.

2-19. (canceled)

20. A method of detecting for capturing and detecting target cells in a sample, the method comprising:
removing red blood cells from a blood sample derived from a subject, wherein the subject is suspected of having cancer or previously diagnosed with cancer;
depositing the sample derived from the subject on a device comprising one or more carbon nanotubes arrays formed with carbon nanotubes, wherein surfaces of the carbon nanotubes are not functionalized; and
detecting presence or absence of circulating tumor cells adhered to the carbon nanotubes, wherein the presence of circulating tumor cells is indicative of cancer.

21.-53. (canceled)

54. A method for capturing and detecting target cells in a sample, the method comprising:
removing, from a sample of a bodily fluid, cells having a settling rate higher than a settling rate of target cells;
depositing the sample on a device comprising carbon nanotubes, wherein surfaces of the carbon nanotubes are not functionalized; and
detecting target cells adhered to the carbon nanotubes.

55. The method according to claim 54, wherein said sample of a bodily fluid is a blood sample, and wherein said cells removed from said sample are red blood cells.

56. The method according to claim 55, wherein said red blood cells are removed from said sample by lyzing the red blood cells or by centrifuging said sample.

57. The method according to claim 54, wherein said target cells are circulating tumor cells, and
wherein said target cells in said sample are from a subject suspected of having cancer or previously diagnosed with cancer.

58. The method according to claim 57, wherein, in said step of detecting, the presence or absence of circulating tumor cells adhered to the carbon nanotubes is indicative of cancer.

59. The method according to claim 54, wherein between 1 and 1500 target cells or circulating tumor cells are captured per milliliter of the bodily fluid.

60. The method according to claim 54, further comprising:
adding a culture medium to the sample, wherein the culture medium is selected based on a target cell or a circulating tumor cell.

61. The method according to claim 54, further comprising:
removing serum from the sample prior to depositing the sample on said device.

62. The method according to claim 54 further comprising:
removing non-adhered cells or material from said device.

63. The method according to claim 62, wherein said removing non-adhered cells or material from the device comprises washing the device.

64. The method according to claim 62, wherein the non-adhered cells comprise white blood cells.

65. The method according to claim 54, further comprising:
counting cells adhered to the carbon nanotubes.

66. The method according to claim 54, further comprising:
collecting the target cells adhered to the carbon nanotubes.

67. The method according to claim 66, further comprising:
characterizing collected target cells, wherein said step of characterizing is selected from the group consisting of genotyping the collected target cells, phenotyping the collected target cells, and combinations thereof, and
wherein said step of phenotyping comprises determining epithelial, mesenchymal, and epithelial-to-mesenchymal transition states of the collected target cells.

68. The method according to claim 54, wherein a surface of the device has an SU8 layer.

69. The method according to claim 54, wherein the device is disposed on a surface of an array of carbon nanotubes.

70. The method according to claim 54, wherein increasing the amount of time elapsed between said step of depositing the sample on the device and said step of detecting improves a capture rate of said target cells.

71. The method according to claim 54, wherein non-functionalized carbon nanotubes are coated in collagen.

72. The method according to claim 54, wherein the density of said plurality of carbon nanotubes is between 1 and 5 nanotubes per micrometer.

73. The method according to claim 54, wherein said sample of a bodily fluid is a saliva or urine sample.

74. (canceled)
